(19) United States
(12) Patent Application Publication
(10) Pub. No.: US 2013/0017250 A1
(43) Pub. Date: Jan. 17, 2013

(54) Methods For High Density Lipoprotein Cholesterol Regulation

(71) Applicants: Henry Ginsberg, Dobbs Ferry, NY (US); Jing Liu, New York, NY (US)

(72) Inventors: Henry Ginsberg, Dobbs Ferry, NY (US); Jing Liu, New York, NY (US)

(73) Assignee: The Trustees of Columbia University in the City of New York, New York City, NY (US)

(21) Appl. No.: 13/550,567

(22) Filed: Jul. 16, 2012

Related U.S. Application Data
(60) Provisional application No. 61/508,601, filed on Jul. 15, 2011.

(51) Int. Cl. A61K 38/44; G01N 21/76; A61P 3/10; C07H 21/04; A61K 9/127; A61P 9/00; G01N 21/17; G01N 21/64

(52) U.S. Cl. 424/450; 435/29; 435/8; 424/94.4; 536/24.1

(57) ABSTRACT

It was discovered that insulin binding to insulin receptors signals the upregulation of expression of the liver enzyme deiodinase 1 (Dio1), which in turn activates the ApoA-1 promoter, thereby thereby increasing ApoA-1 expression (primarily in the liver), that in turn raises the levels of plasma ApoA-1, the major and necessary protein in HDLC. Certain embodiments of the invention are directed to methods for increasing circulating HDLC levels in an animal by administering therapeutically effective amounts of Dio1, or by increasing the level of Dio1 through gene therapy.

### STATEMENT OF GOVERNMENTAL INTEREST

This invention was made with Government support under grants R01 HL55638 and R01 HL73030 awarded by NHLBI. The Government has certain rights in the invention.

### BACKGROUND OF THE INVENTION

1. Field of the Invention

Embodiments of the invention relate to methods of screening for agents that increase deiodinase 1 promoter activity and deiodinase 1 biological activity or expression, or ApoA-1 promoter activity and ApoA-1 expression, and to methods for raising plasma HDLC or ApoA-1 levels in a subject in need of such treatment by administering therapeutically effective amounts of deiodinase 1 or biologically active fragment or variant thereof.

2. Description of the Related Art

Diabetes mellitus is a family of disorders characterized by chronic hyperglycemia and the development of long-term complications. This family of disorders includes type 1 diabetes, type 2 diabetes, gestational diabetes, and other types of diabetes. In 2006, cardiovascular disease affected 81.1 million people in the U.S. and kills 17.1 million people per year worldwide. People with lower levels of high density lipoprotein cholesterol (HDLC) and apolipoprotein A-I (ApoA-1) have a higher risk of cardiovascular disease. (1-6) Low levels of HDLC are common in individuals who are insulin resistant (IR), e.g., with metabolic syndrome, and type 2 diabetes mellitus (T2DM). Although much of the low HDLC phenotype is related to high triglycerides (TG) through cholesterol ester transfer protein (CETP) (7), this relationship actually accounts statistically for less than 50% of the variability in HDL levels (8; 9). Importantly, very little work has been reported regarding insulin signaling or action and the levels of either HDLC or ApoA-1 (10-12).

Atherosclerosis and its associated coronary heart disease is the leading cause of death in the industrialized world. Risk for development of coronary heart disease has been shown to be strongly correlated with certain plasma lipid levels. Lipids are transported in the blood by lipoproteins. The general structure of lipoproteins is a core of neutral lipids (triglyceride and cholesterol ester) and an envelope of polar lipids (phospholipids and non-esterified cholesterol). There are three different major classes of plasma lipoproteins with different core lipid content: the low density lipoprotein (LDL) which is cholesteryl ester (CE)-rich; high density lipoprotein (HDL) which is also cholesteryl ester (CE) rich; and the very low density lipoprotein (VLDL) which is triglyceride (TG) rich. The different lipoproteins can be separated based on their different flotation density or size.

High LDL-cholesterol (LDL-C) and triglyceride levels are positively correlated, while high levels of HDL-cholesterol (HDL-C) are negatively correlated with the risk for developing cardiovascular diseases.

No wholly satisfactory HDL-elevating therapies exist. As a result, there is a significant unmet medical need for a well-tolerated agent which can significantly elevate plasma HDL levels for the treatment and/or prophylaxis of atherosclerosis, peripheral vascular disease, dyslipidemia, hyperbetalipoproteinemia, hypoalphalipoproteinemia, hypercholesterolemia, hypertriglyceridemia, familial hypercholesterolemia, cardiovascular disorders, angina, ischemia, cardiac ischemia, stroke, myocardial infarction, stroke, reperfusion injury, angioplastic restenosis, hypertension, and vascular complications of diabetes, obesity or endotoxemia.

SUMMARY OF INVENTION

Certain embodiments of the invention are directed to methods for identifying test agents capable of increasing Dio1 or ApoA-1 promoter activity, for example, a method comprising a. providing a first control population and a first test population of mammalian cells genetically engineered to express a nucleic acid encoding a deiodinase 1 promoter or ApoA-1 promoter Construct B identified by SEQ ID NO: 24, which promoter is operatively linked to a reporter protein that can be visualized under conditions that permit the cells in the population to express the reporter protein, b. contacting the first test population with the a test agent, c. determining the amount of visualized reporter protein in the first control population and the first test population, and d. if the determined amount in the first test population is higher than the determined amount in the first control population, then identifying the test agent as one that increases the activity of the respective deiodinase 1 promoter or ApoA-1 promoter. Another embodiment is the method of claim 1, wherein if the test agent is identified as one that increases the activity of either the deiodinase 1 promoter or ApoA-1 promoter B construct, then e. providing a second control and a second test population of the cells that have been transfected with a nucleic acid encoding deiodinase 1 or ApoA-1 protein or a biologically active fragment or variant that has at least 70% sequence identity therewith, which encoding nucleic acid is operatively linked to reporter a reporter protein that can be visualized under conditions that permit the cells to express the reporter protein, f. contacting the second test population with the test agent, g. determining the amount of visualized reporter protein in the second control population and the second test population, and h. if the determined amount in the second test population is higher than the determined amount in the second control population, then identifying the test agent as one that increases deiodinase 1 or ApoA-1 protein expression by increasing activity of the respective promoter. In preferred embodiments the provided control and test populations exhibit reduced insulin receptor expression or biological activity.

In some embodiments the reporter protein is a fluorescent protein and in some the reporter protein is a member selected from the group comprising alkaline phosphatase, horseradish peroxidase, urease, beta galactosidase, and chloramphenicol acyltransferase.

In some embodiments of the assay the insulin receptor gene in the cells has been knocked out, or it has been suppressed with an inhibitory oligonucleotide.

In an embodiment wherein the test agent is identified as one that increases either deiodinase 1 expression or ApoA-1 expression, the test agent is administered to an animal it is determined whether the agent increases plasma HDLC and/or ApoA-1 levels. In preferred embodiments of the assays the cells are liver cells such as the liver cells are from human hepatoma cell line HepG2 or rat hepatoma cell line McARH7777, and the biological sample is a blood sample, plasma or a tissue sample.

Other embodiments are directed to therapies for increasing the levels of plasma high density lipoprotein cholesterol (HDLC) and/or plasma ApoA-1 levels in a subject having lower than desired levels by administering to the subject deiodinase 1, or a biologically active protein or variant that has at least 70% identity with the amino acid sequence of deiodinase 1, in a therapeutically effective amount that increases the plasma levels of HDLC and or ApoA-1. In certain embodiments the subject in need of such treatment is an animal having type 2 diabetes, cardiovascular disease or a disorder associated with impaired or defective insulin signaling, and the deiodinase 1 is formulated to optimize delivery to the liver.

Another embodiment is directed to a pharmaceutical formulation comprising human deiodinase 1 or a biologically active protein or variant that has at least 70% identity with the amino acid sequence of deiodinase 1, formulated in liposomes and targeted to the liver. Another embodiment is directed to an oligonucleotide identified by SEQ ID NO: 24.

### BRIEF DESCRIPTION OF THE DRAWINGS

The present invention is illustrated by way of example, and not by way of limitation, in the figures of the accompanying drawings and in which:

FIG. 1 Analysis of FPLC shows that 5 Month LIRKO mice have reduced HDLC compared with controls.

FIG. 2 Analysis of FPLC 6 days after insulin receptors were reduced by Ad-Cre injection into insulin receptor floxed mice showed reduced HDLC levels (compared to floxed mice receiving Ad-LacZ.

FIG. 3 Microarray data demonstrating that knockdown of hepatic Insr in floxed mice by adenovirus carrying cDNA for albumin-Cre dramatically decreased Dio1 mRNA

FIG. 4 qPCR data confirming microarray results that knockdown of hepatic InsR decreased ApoA-I and several other lipoprotein related genes.

FIG. 5A Expression of Dio1 mRNA in LIRKO mice increased significantly following administration of an adenovirus with cDNA for a constitutively active Akt compared with control. FIG. 5B Restoration of insulin signaling via AKT also restored HDLC.

FIG. 6 siRNA for insulin receptor markedly reduces mRNA levels of both insulin receptors and Dio1 in McArdle RH7777 rat hepatoma cells.

FIG. 7 Dio1 mRNA levels increased 100% in LIRKO mice receiving an adenovirus carrying Dio1 cDNA compared with LIRKO mice receiving an adenovirus carrying GFP cDNA.

FIG. 8 Levels of ApoA-1 mRNA increased in LIRKO mice receiving the Dio1 adenovirus compared with GFP.

FIG. 9 HDL cholesterol went up in LIRKO mice after Ad-Dio1 treatment compared to mice receiving Ad-GFP.

FIG. 10 siRNA knock-down of Insulin Receptors reduced expression of both Dio1 and ApoA-1 in McArdle RH7777 cells.

FIG. 11 Knock down of either InsR or Dio 1 in McArdle RH7777 cells with siRNA reduced the activity of a rat ApoA-1 promoter, as assessed by a luciferase reporter.

FIG. 12 ApoA-1 mRNA was significantly decreased in livers of Dio1-KO mice.

FIG. 13 Knockdown of Insr by siRNA decreased ApoA-1 promoter activity in both McArdle RH7777 cells and HepG2 cells

FIG. 14 Treatment of HepG2 cells with an AKT1/2 inhibitor dramatically decreased Dio1 promoter activity.

FIG. 15 Knockdown of Insr by siRNA decreased Dio1 promoter activity in HepG2 cells.

FIG. 16 HepG2 cells were co-transfected with siRNA-Insr or siRNA-Ctrl and ApoA-1(−256bp)-Luc plasmid. Later, cells were infected with Ad-Dio1 or Ad-GFP. Expression of Dio1 (by adenoviral infection) reversed the reduction in ApoA-1 promoter activity in HepG2 cells treated with siRNA-Insr.

FIG. 17A Schematic representation of human ApoA-1 promoter constructs. Construct A and C contain HREs which bind nuclear receptor superfamily members. Construct B binds C/EBP and other transcription factors. FIG. 17B HepG2 cells were transfected with ApoA-1-Luc-ABC, -BC or -C. These regions have been previously mapped and have distinct complements of response elements. 6 hrs later, the cells were infected with ad-sh-Insr or Ad-sh-GFP. Results show that insulin signaling regulates human ApoA-1 promoter activity by acting on the B region of the promoter (−192-−128 bp, relative to transcription start site).

### DETAILED DESCRIPTION

Before the present compositions and methods are described, it is to be understood that this invention is not limited to the particular processes, compositions, or methodologies described, as these may vary. The terminology used in the description is for the purpose of describing the particular versions or embodiments only, and is not intended to limit the scope of the present invention. Unless otherwise defined, all technical and scientific terms used herein have the same meanings as commonly understood by one of ordinary skill in the art. Although any methods and materials similar or equivalent to those described herein can be used in the practice or testing of embodiments of the present invention, the preferred methods, devices, and materials are now described. All publications mentioned herein, are incorporated by reference in their entirety. Nothing herein is to be construed as an admission that the invention is not entitled to antedate such disclosure by virtue of prior invention.

### DEFINITIONS

“Hormone response element (HRE)” means a short sequence of DNA within the promoter of a gene that is able to bind a specific hormone receptor. A promoter may have many different response elements, allowing complex control to be exerted over the level and rate of transcription.

“CCAAT-enhancer-binding proteins (or C/EBPs)” means a family of transcription factors, composed of six members called C/EBPα to C/EBPζ. They promote the expression of certain genes through interaction with their promoter. Once bound to DNA, C/EBPs can recruit so-called co-activators (such as CBP, see ref. 2) that, in turn, can open up chromatin structure, or recruit basal transcription factors.

“Transfection of an animal cell” means the process of deliberately introducing nucleic acids into cells. The term is used notably for non-viral methods in eukaryotic cells. Transfection typically involves opening transient pores or “holes” in the cell membrane, to allow the uptake of material, typically nucleic acids. Transfection can be carried out for example using calcium phosphate.

“A genetically engineered cell or organism” means one generated through the introduction of recombinant DNA and is considered to be a genetically modified cell or organism.

“Apolipoprotein A-1 (ApoA-1)” means a protein that in humans is encoded by the ApoA-1 gene and it is the major protein component of high density lipoprotein (HDL).

“Liver-specific insulin receptor knockout (LIRKO) mice” means mice that do not express insulin receptors and in which Dio1, ApoA-1 and plasma HDLC levels are low.

“Type 1 iodothyronine deiodinase (D1 liver selenoenzyme deiodinase type 1 (D1), deiodinase 1 (Dio1), iodide peroxidase, monodeiodinase and isozymes of Dio1” mean a peroxidase enzyme that is mainly located in the liver that is involved in the conversion of T4 (thyroxine) into T3 (triiodothyronine) by the deiodinase enzyme in target cells.

“A subject with low levels of plasma high density lipoprotein cholesterol (HDLC)” means a subject whose respective levels are lower than normal or lower than desired; such a subject is in need of treatment to raise the HDLC level.

“Prophylactically effective amount” means an amount of a therapeutic agent, which, when administered to a subject, will have the intended prophylactic effect e.g., preventing or delaying the onset (or reoccurrence) lower than desirable levels of plasma HDLC. The full prophylactic effect does not necessarily occur by administration of one dose and may occur only after administration of a series of doses. Thus, a prophylactically effective amount may be administered in one or more administrations.

“Subject” means a patient or an animal, including mammals, e.g., humans, dogs, cows, horses, kangaroos, pigs, sheep, goats, cats, mice, rabbits, rats, and transgenic non-human animals.

“Therapeutically effective amount” means an amount of a therapeutic agent that achieves an intended therapeutic effect in a subject, e.g., increasing plasma HDLC or plasma ApoA-1, preferably to normal levels. The full therapeutic effect does not necessarily occur by administration of one dose and may occur only after administration of a series of doses. Thus, a therapeutically effective amount may be administered in one or more administrations.

“Treating” means taking steps to obtain beneficial or desired results, including clinical results, such as mitigating, alleviating or ameliorating one or more symptoms of a disease; diminishing the extent of disease; delaying or slowing disease progression; ameliorating and palliating or stabilizing a metric (statistic) of disease. In this case the disease is lower than desirable levels of plasma HDLC. “Treatment” refers to the steps taken.

A new pathway has been discovered for upregulating plasma HDLC and ApoA-I levels through insulin signaling in the liver. It was discovered that insulin binding to insulin receptors signals the upregulation of expression of the enzyme deiodinase 1 (Dio1), which in turn activates the ApoA-1 promoter, thereby increasing ApoA-1 expression (primarily in the liver) that in turn raises the levels of plasma ApoA-I, the major and necessary protein in HDLC. Certain experiments described herein were done using liver-specific insulin receptor knockout (LIRKO) mice that do not express insulin receptors, and in which Dio1, ApoA-1 and plasma HDLC levels are low. The results described in detail in the figures and examples show that restoration of Dio1 expression, for example through transfection with an adenovirus carrying a gene for the enzyme, restored normal HDLC levels and raised the level of ApoA-1 in LIRKO mice. Adenoviral Gene Therapy, Stephan A. Vorburger, et al., The Oncologist 2002; 7:46-59.

Certain embodiments of the invention are directed to methods for increasing circulating HDLC levels in an animal by administering therapeutically effective amounts of Dio1, or by increasing the level of Dio1-through gene therapy.

Other embodiments are directed to high throughput screening methods to identify compounds that increase Dio1 or ApoA-1 promoter activity thereby increasing the expression of Dio1 and/or ApoA-1, respectfully.

### SUMMARY OF THE RESULTS

It has been previously reported that liver-specific insulin receptor knockout (LIRKO) mice had markedly reduced plasma HDLC levels, and that an insulin-resistant mouse with defective PI-3K signaling (PI-3K knockout mice) also had low plasma HDLC. However, when LIRKO mice were treated with an adenovirus containing cDNA for a constitutively active form of Akt, which restores liver insulin signaling despite the absence of liver insulin receptors, the level of plasma HDLC increased significantly compared to LIRKO mice given a control adenovirus. (13). Plasma triglyceride levels were also shown to be reduced in LIRKO mice (13).

The experiments described herein focused on identifying the mechanism through which the lack of insulin receptors in the liver causes reduced plasma ApoA-I and HDLC, or stated another way, the mechanism by which signaling through insulin receptors maintains normal plasma ApoA-I and HDLC levels.

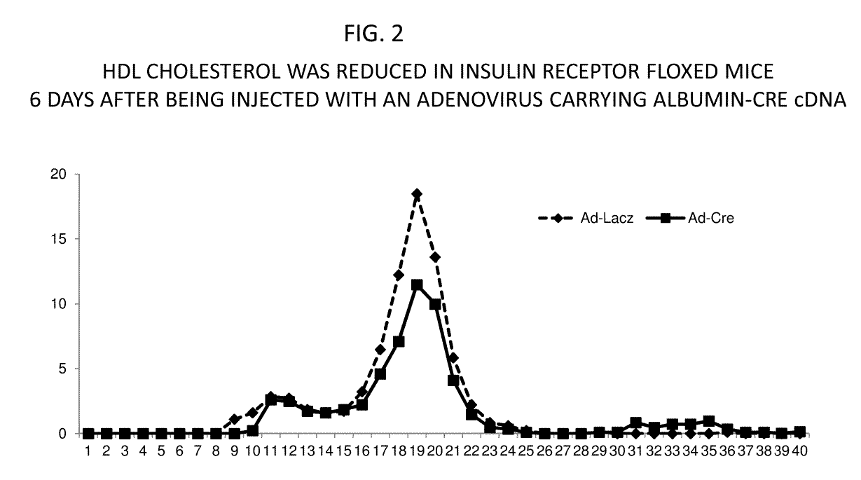

When mice with floxed insulin receptors were treated with an adenovirus containing cDNA for the Cre enzyme controlled by the albumin promoter (for complete hepatic specificity), there was a significant loss of hepatic insulin receptors over the next week. Data not shown. In association with the acute loss of hepatic insulin receptor, there was a reduction in HDLC, demonstrated by FPLC. FIG. 2; Example 1. This model functioned like LIRKO mice with respect to insulin signaling and HDLC.

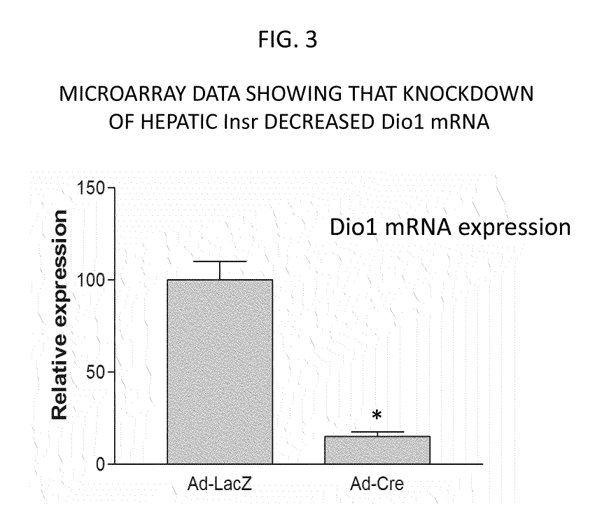

Microarrays (confirmed by qPCR) performed on these mice revealed that knockdown of hepatic insulin receptors also markedly reduced hepatic expression of several apolipoprotein genes, including ApoA-1 (the major protein component of high density lipoprotein (HDL) in plasma) (FIGg. 4) and the enzyme deiodinasel (FIG. 3). Deiodinasel (Dio1) is an enzyme that is mainly expressed in liver where it converts T4 to T3 or rT3, and rT3 to T2 (14-18). Example 1.

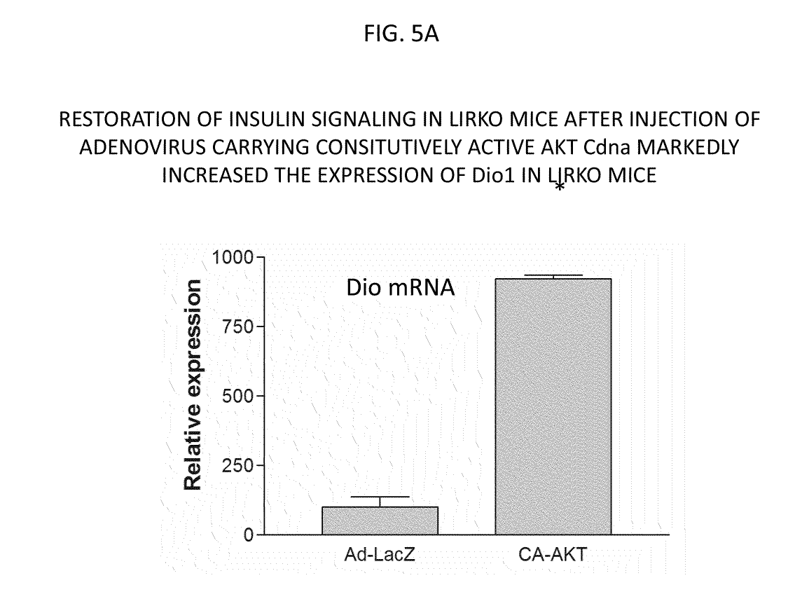

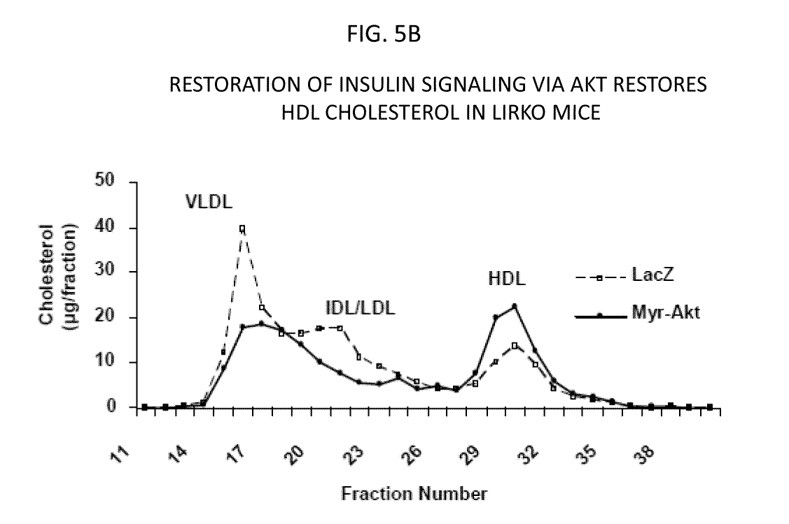

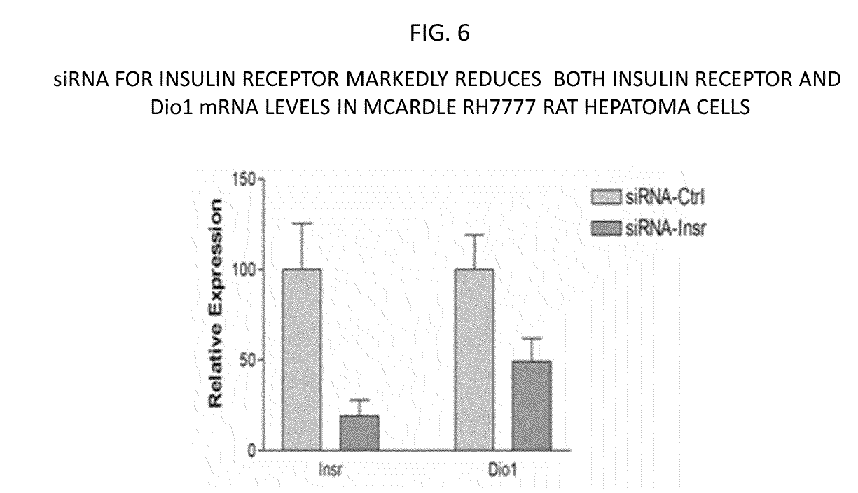

Insulin signaling was restored by administering an adenovirus with cDNA for a constitutively active Akt to 16 week old LIRKO mice (as was shown in the Biddinger paper above). Importantly, the restoration of insulin signaling markedly increased the expression of Dio1. FIG. 5; Example 2, and in McArdle RH7777 rat hepatoma cells, treatment with an siRNA that blocked the expression of insulin receptors resulted in marked reductions in both insulin receptor and Dio1 mRNA levels. FIG. 6; Example 3.

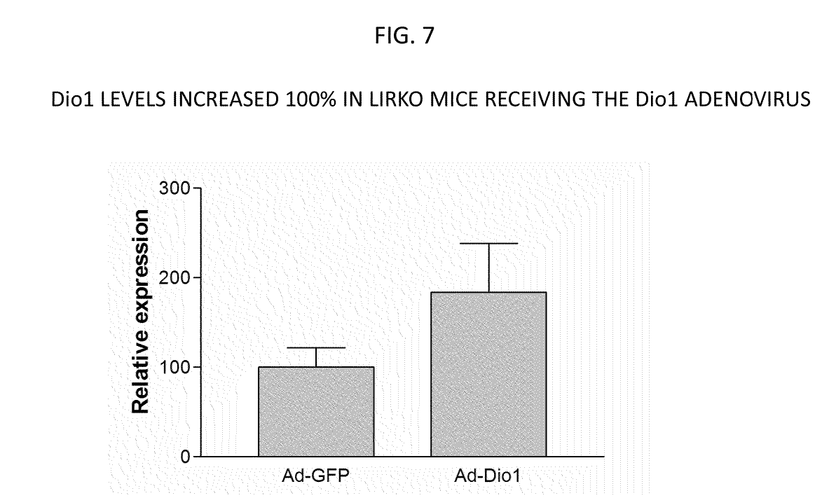

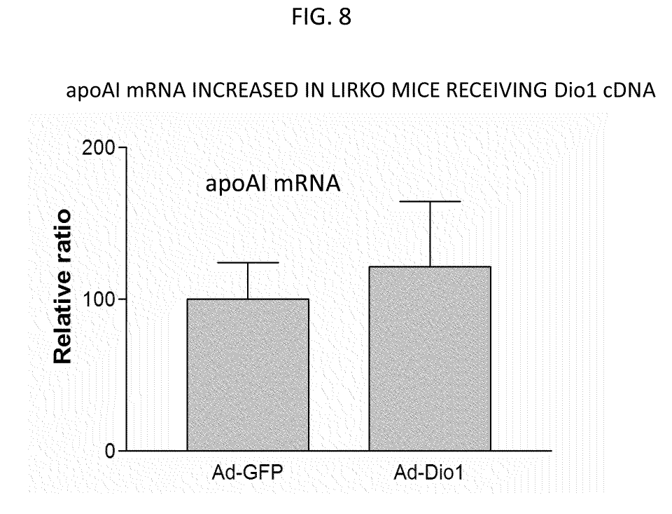

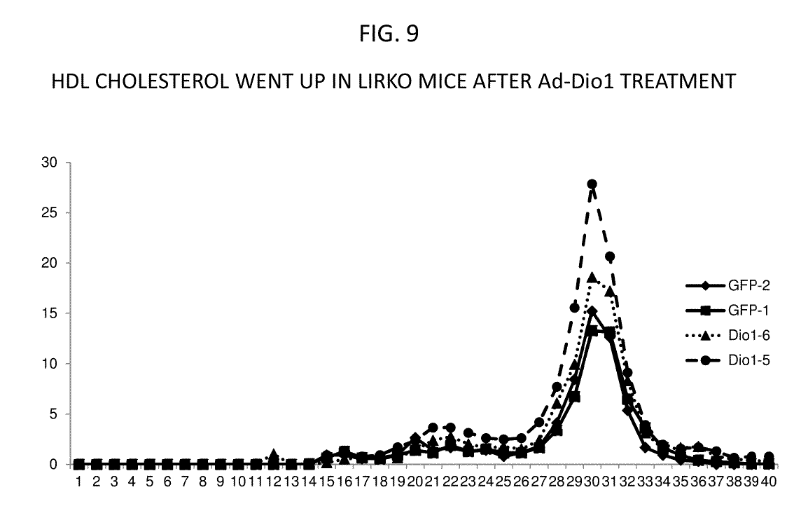

To investigate whether insulin signaling through Dio1 is involved in the regulation of HDLC levels, an adenovirus containing cDNA for Dio1 was administered to LIRKO mice. Diol levels were increased 100% in mice 12 days after receiving the Dio1 adenovirus (FIG. 7), and there was a related trend toward increased ApoA-1 mRNA (FIG. 8). Importantly, HDLC levels, assessed by FPLC, increased in each of the mice injected with the Dio1 adenovirus showing a causal relationship between increased Dio1 expression and increased plasma HDLC. FIG. 9 Additionally, ApoA-1 in the HDL fractions (isolated from the FPLC) was also increased in mice receiving the Dio1 adenovirus. Data not shown.

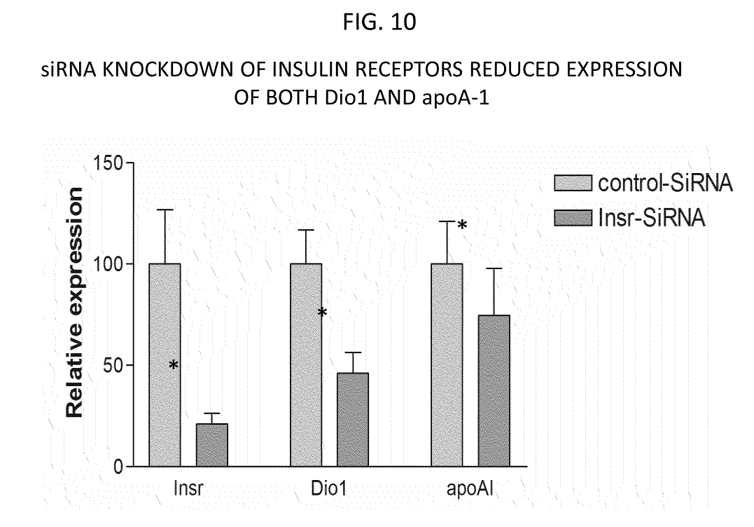

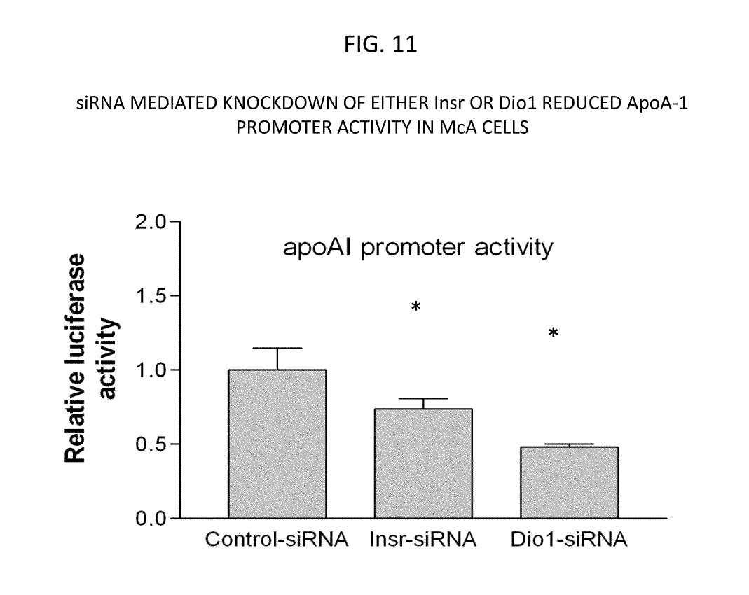

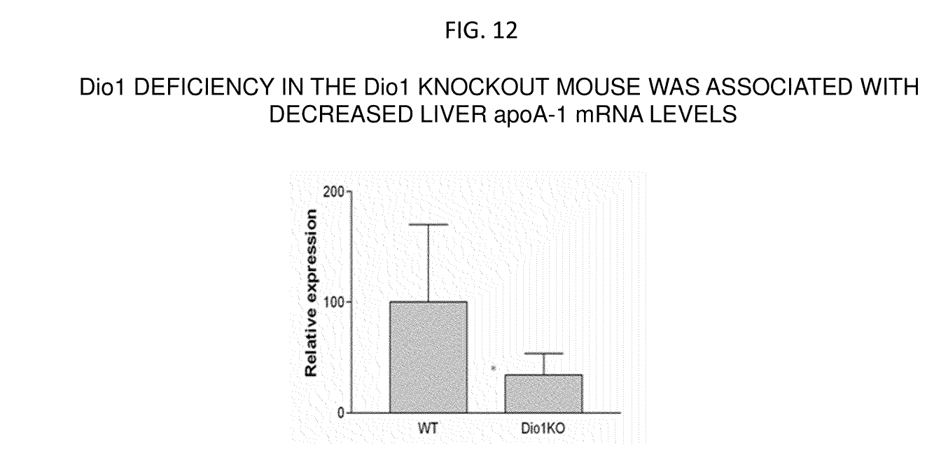

When insulin receptors in McArdle RH7777 hepatoma cells were knocked down with an siRNA, the expression of ApoA-1 (as well as Dio1) was significantly reduced. FIG. 10. Furthermore, a direct knockdown of Dio1 with a siRNA reduced the activity of the rat ApoA-1 promoter. These data indicate that normally, there is a direct effect of insulin signaling causing an increase in Dio1 expression, which in turn causes an increase in transcription of the ApoA-1 gene through Dio1 activating the ApoA-1 promoter. FIG. 11. As shown in FIG. 12, ApoA-1 mRNA levels were significantly reduced in livers from Dio1 knockout mice. Example 5.

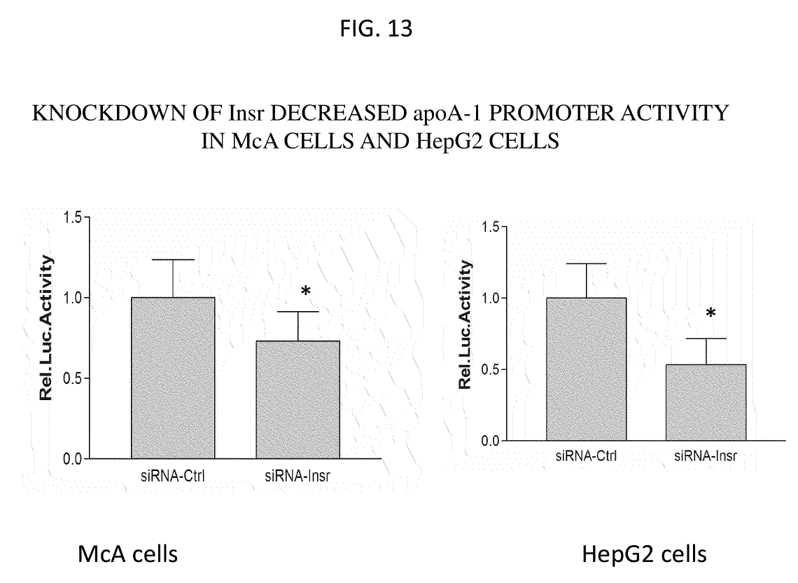

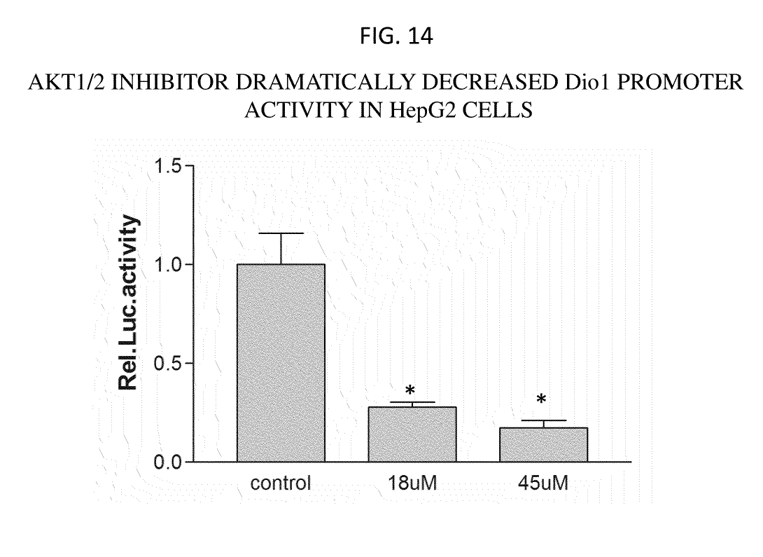

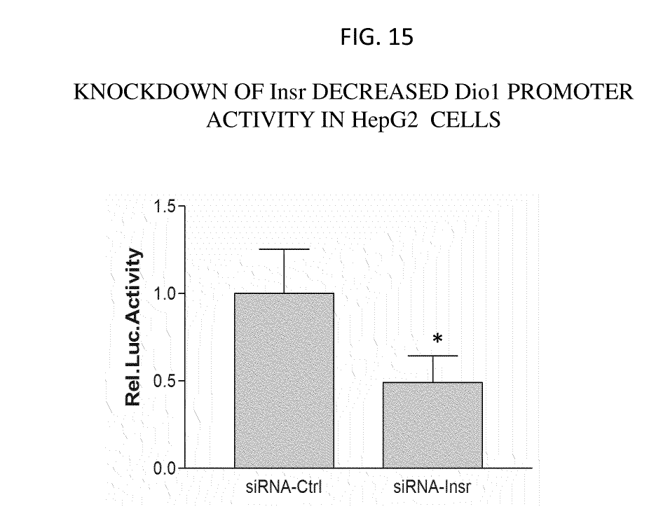

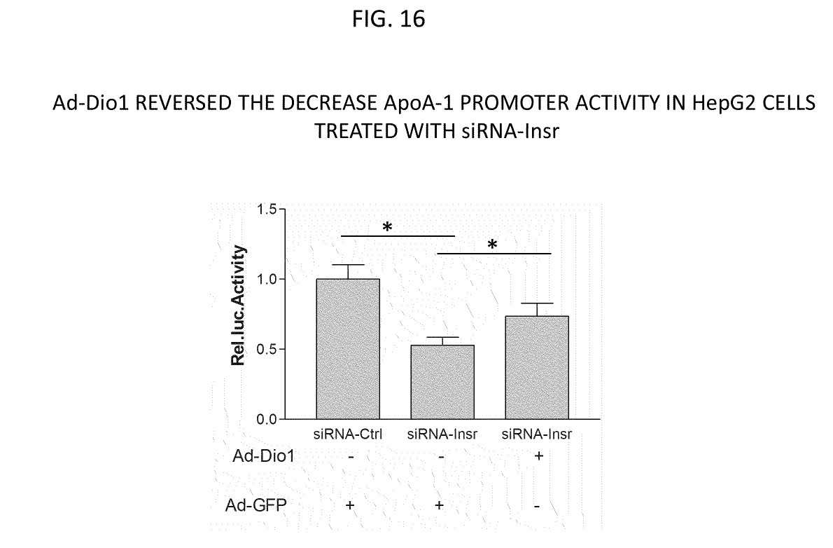

Knockdown of Insr decreased ApoA-1 promoter activity in McArdle 7777 cells and in HepG2 cells (FIG. 13), and decreased Dio1 promoter activity in HepG2 cells. FIG. 15. AKT1/2 inhibitor dramatically decreased ApoA-1 promoter activity in hepG2 cells (FIG. 14). However, when HepG2 cells were co-transfected with siRNA-Insr (or siRNA-Ctrl) and an ApoA-1(−256 bp)-Luc plasmid; and then infected with Ad-Dio1 (or Ad-GFP), Ad-Dio1 expression was able to reverse the inhibition of ApoA-1 promoter activity. FIG. 16.

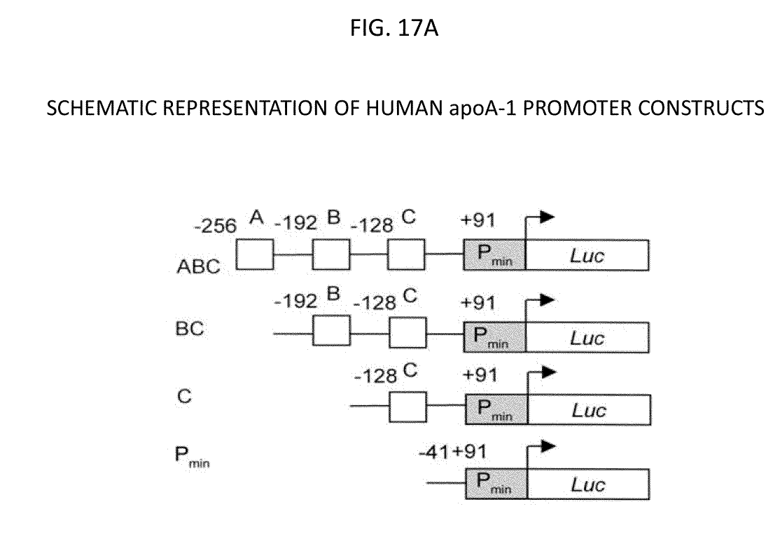

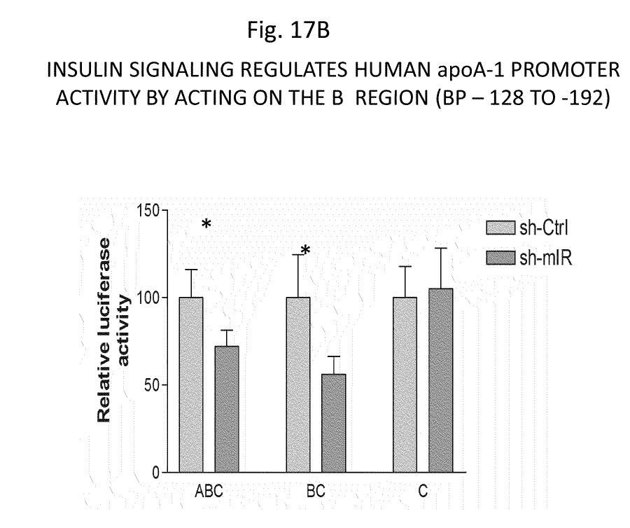

Although there have been studies reporting reduced Dio1 activity (19) and Dio1 mRNA levels (20) in streptozotocin (STZ)-treated rats, there exist no published data indicating that insulin signaling directly regulates Dio1. More importantly, although the ApoA-1 promoter has thyroid response elements (TREs), there are no studies linking Dio1 to the regulation of ApoA-1 or HDLC levels. A schematic representation of human ApoA-1 promoter (FIG. 17A) shows three constructs: Construct A (SEQ ID NO. 23) and Construct C (SEQ ID NO. 25) contain HREs which bind nuclear receptor superfamily. Construct B (SEQ ID NO. 24) binds several other transcription factors including C/EBP, and consists of −192-−128bp relative to transcription start site of the ApoA-1 promoter. Construct B contains a region that is necessary for activation of the human ApoA-1 promoter activity by insulin signaling (FIG. 17B). Without being bound by theory, others have reported that the insulin binding site on the ApoA-1 promoter is significantly upstream (>400 by from the start of translation) from Construct B nucleotide. J Biol Chem. 1998 Jul 24; 273(30):18959-65. Murao K, et al. however, insulin may not bind directly to DNA—even the reported insulin. Instead there is more likely an insulin responsive element and the signal starts with insulin at the receptor but the binding molecule is something other than insulin itself. An embodiment of the invention is also directed to the B Construct.

Based on the results of experiments described above and in the Examples, a new pathway has been identified showing that insulin regulates Dio1 expression by activating the Dio1 promoter, and Dio1 in turn regulates the transcription of ApoA-1 by activating the ApoA-1 promoter, resulting in normal plasma HDLC levels. Certain embodiments are directed to new therapies that raise plasma HDLC levels in individuals low plasma HDLC, such as certain individuals having type 2 diabetes, cardiovascular disease or a disorder associated with impaired or defective insulin signaling, by administering therapeutically effective amounts of Dio1 or a biologically active fragment or variant thereof, or using gene therapy to introduce the Dio1 gene, targeted preferably to the liver.

Certain other embodiments are directed to high throughput screening methods to identify compounds that increase Dio1 promotor activity or Dio1 expression, or ApoA-1 promoter activity.

### EXAMPLES

### Example 1

### Loss of Hepatic Insulin Receptors Was Accompanied By An Unexpected Reduction In HDL Cholesterol And An Increase In Dio1

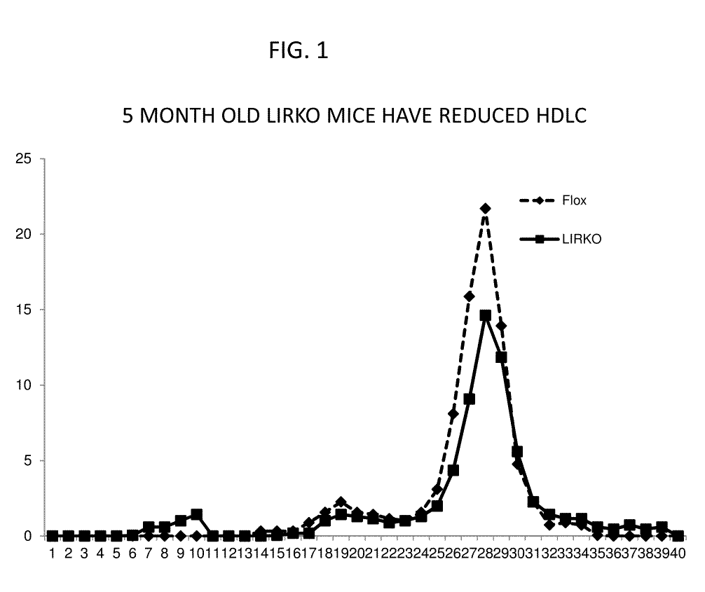

FIG. 1 shows that LIRKO mice had reduced levels of HDL cholesterol levels by FPLC. 200 μl of serum was pooled from 4 h fasted LIRKO and Floxed mice and subjected to fast protein liquid chromatography (FPLC) using a single Superose 6 column (GE Healthcare). Proteins were eluted at 0.30 ml/min (elution buffer: 150 mM NaCl/l mM EDTA, pH 8). Forty fractions (0.5 ml) were collected and total cholesterol content in each fraction was determined by enzymatic kit (Wako diagnostic).

LIRKO mice also had reduced levels of plasma ApoA-1. 0.5 μl of serum from 4 h fasted 4 month old LIRKO and Floxed mice were run on SDS-polyacrylamide gel electrophoresis, protein bands on the gel were transferred to nitrocellulose membrane (Bio Rad). Incubation of anti-mouse ApoA-1 antibody (Calbiochem) with the membrane was performed in TBST including 0.1% Tween 20 and 2% nonfat milk at 4 C overnight. Detection of the immune complexes was carried out by ECL Western Blotting Substrate (Thermo Scientific). Data not shown. This is because blots don't show up well in patents. ApoA-1 antibodies are also available from Santa Cruz company ApoA-1 (FL-267) Antibody: sc-3008 against human, mouse and rat.

In another experiment, mice that were floxed at the insulin receptor genes were treated with an adenovirus containing cDNA for the Cre enzyme, which is controlled by the albumin promoter. Insulin receptor was decreased by 90% after injection of Ad-Cre into LIRKO mice. When 12 week old mice with floxed insulin receptor genes were injected with adenovirus-encoding recombinase Cre (under the control of a liver-specific albumin promoter for complete hepatic specificity), there was a significant loss of hepatic insulin receptors over the next week. In these experiments the adenovirus was administered via the femoral vein at a dosage of 1×10 pfu. An adenovirus containing β-galactosidase (LacZ) was used as a control. Mice were sacrificed at days 3, 6, and 11 after adenovirus injection and livers were snap-frozen in liquid nitrogen. 50 ug of liver homogenate proteins were subjected to 8% SDS-PAGE and electrophoretically transferred to nitrocellulose membrane (Bio-Rad). The membrane was then incubated with anti-mouse insulin receptor β polyclonal antibody (Santa Cruz) at 4 C overnight. The blot was treated with HRP-conjugated goat anti-Rabbit IgG for 1 h and visualized by chemiluminescence (ECL; Thermo Scientific). Data not shown.

Importantly, it was observed that the loss of hepatic insulin receptors was accompanied by an unexpected reduction in HDL cholesterol, demonstrated by FPLC. FIG. 2. 12 week old mice with floxed insulin receptor genes were injected with adenovirus encoding recombinase Cre under the control of a liver-specific albumin promoter as described above. β-galactosidase (LacZ) was used for a control. Blood was collected on day 6 after adenovirus injection. 200 μl of pooled serums from 3 mice treated with either Ad-Cre or Ad-LacZ were subjected to fast protein liquid chromatography (FPLC) using a single Superose 6 column (GE Healthcare). Proteins were eluted at 0.30 ml/min (elution buffer: 150 mM NaCl/l mM EDTA, pH 8). Forty fractions (0.5 ml) were collected and total cholesterol content in each fraction was determined by enzymatic kit (Wako diagnostic).

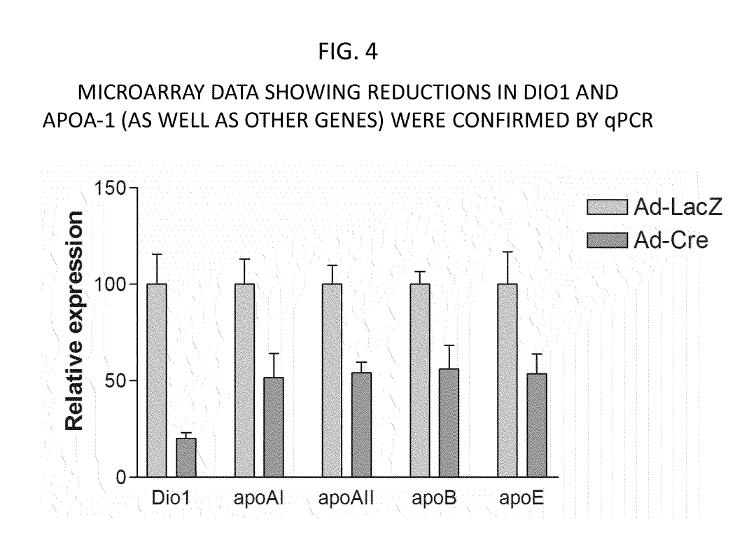

Microarray analysis further indicated that knockdown of hepatic insulin receptors also markedly reduced hepatic expression of deiodinase 1 (Dio1) as shown in FIG. 3. Liver samples from floxed mice that had been treated with Ad-Cre (day 6) and Ad-LacZ (day 6) were sent to Ocean Ridge Biosciences for cDNA microarray analysis. Microarray analyses were performed using MEEBO (The Mouse Exonic Evidence Based Oligonucleotide) DNA array. The oligonucleotide set consists of 38,784 70-mer probes that were designed using a transcriptome-based annotation of exonic structure for genomic loci. Genes involved in insulin signaling pathway and lipid metabolism were examined for over-representation of differentially expressed genes based on fold change criteria. Only those genes that had a twofold change in expression were included in this analysis. Expression of deiodinase 1 decreased dramatically in these animals. The microarray data were confirmed by qPCR as shown in FIG. 4.

Furthermore, there was also a significant reduction in a number of other lipoprotein-related genes as was seen both on the array and by qPCR. One such protein that was significantly reduced was ApoA-1, the major and crucial protein in HDL metabolism. To make this determination, total RNA was isolated from the same samples sent for microarray analysis using TRIzol reagent (Invitrogen). First-strand cDNA was synthesized from 4 μg of total RNA using Oligo(dT) primers and SuperScript II reverse transcriptase (Invitrogen). Real time PCR was performed in 25 ul of total volume with the use of Brilliant SYBR green qRT-PCR master mix (Agilent Technologies) using the ABI Prism 7700 Sequence Detection System (Applied Biosystems). Primers were obtained from Invitrogen. The mRNA levels were normalized by housekeeping gene cyclophilin. Primers (forward, reverse) used for this study were as follows:

murine (m)-deiodinase 1,
(SEQ ID NO: 1)
CCCCTGGTGTTGAACTTTG,
(SEQ ID NO: 2)
TGTGGCGTGAGCTTCTTC;
mApo-AI,
(SEQ ID NO: 3)
TGTGTATGTGGATGCGGTCA,
(SEQ ID NO: 4)
ATCCCAGAAGTCCCGAGTCA;
mApo-AII,
(SEQ ID NO: 5)
AATGGTCGCACTGCTGGTCA,
(SEQ ID NO: 6)
TTGGCCTTCTCCATCAAATC;
mapoB,
(SEQ ID NO: 7)
ATGGGAAGAAACAGGCTTGA,
(SEQ ID NO: 8)
TTCTGTCCCACGAATTGACA;
mapoE,
(SEQ ID NO: 9)
ACCGCTTCTGGGATTACCTG,
(SEQ ID NO: 10)
GCTGTTCCTCCAGCTCCTTT;
mcyclophilin,
(SEQ ID NO: 11)
GGAGATGGCACAGGAGGAA,
(SEQ ID NO: 12)
GCCCGTAGTGCTTCAGCTT;

The thermo cycling protocol for reverse transcriptase-polymerase chain reaction (RT-PCR) amplification were initial denaturation for 1 minute at 94° C. followed by 40 cycles of 30 sec at 94° C., 30 sec at 60° C., and 45 sec at 72° C.

### Example 2

### Insulin Signaling Was Restored By Overexpression of Akt, Which Signaling In Turn Increased Dio1 Expression

FIG. 7 shows that insulin signaling was restored by administering an adenovirus with cDNA for a constitutively active Akt to 16 week old LIRKO mice (as described in Biddinger et al. above). Restoration of insulin signaling markedly increased the expression of Dio1 as measured by qPCR. In these experiments, 16 week old LIRKO mice were injected intravenously with adenovirus encoding either a constitutively active form of Akt (myr-Akt) or LacZ. Mice were sacrificed and livers were collected on day 6 after injection. Total RNA were extracted, then reverse transcription and qPCR were performed as described above (FIG. 5).

### Example 3

### Reduction of Insulin Receptor mRNA With siRNA Also Reduced Dio1 mRNA

In McArdle RH7777 rat hepatoma cells, it was observed that treatment with an siRNA for the insulin receptor resulted in marked reductions in both insulin receptor and Dio1 mRNA levels as shown in FIG. 6. In these RNA interference transfections, McArdle RH7777 rat hepatoma cells (cultured with DMEM, 10% FBS, 10% horse serum, 1% p/s) were seeded in six-well plates at 2.5×10⁵ cells/well. A pool (40 nM final concentration) of two individual Mission siRNA oligonucleotides (SASI_Rn02_{—}00261274, siRNA1, M SASI_Rn02_{—}00261275, siRNA2, Sigma-Aldrich) was transfected into McArdle RH7777 (McA) cells using nanoparticle-based siRNA transfection reagent (N-TER™, Sigma-Aldrich) 16 h later. Mission siRNA Universal Negative Control (#1; Sigma-Aldrich) was used as a negative control. Cells were collected at 36-48 h after transfection. Total RNA were extracted, then reverse transcription and qPCR were performed as mentioned above (FIG. 6).

Mission siRNA SASI_Rn01_{—}00118857 for Dio1 is:

(SEQ ID NO: 13)
5′GAUUGAAAUCCGUUAAUAU[dT][dT]
(SEQ ID NO: 14)
5′AUAUUAACGGAUUUCAAUC[dT][dT]

Mission siRNA SASI_Rn01_{—}00118856 for Diol is:

| | |
|---|---|
| 5′ CUCAUGAUGAUGACGUCAA[dT] | (SEQ ID NO: 15) |
| 5′ UUGACGUCAUCAUCAUGAG[dT] | (SEQ ID NO: 16) |

Primers (forward, reverse) used for this study were as follows:

Rat (r)-Insulin receptor,
(SEQ ID NO: 17)
ATGGGCTTCGGGAGAGGAT,
(SEQ ID NO: 18)
GGATGTCCATACCAGGGCAC;
rDeiodinase 1,
(SEQ ID NO: 19)
CCCCTGGTGTTGAACTTTG,
(SEQ ID NO: 20)
TGTGGCGTGAGCTTCTTC.

### Example 4

### Overexpression of Dio1 In LIRKO Mice Increased ApoA-1 Expression And HDL Levels

To investigate the pathway from insulin signaling to Dio1 and then to ApoA-1, an adenovirus containing cDNA for Dio1 to LIRKO mice was administered intravenously. An adenovirus containing a GFP construct was used as a control. It was found that Dio1 levels increased 100% in mice receiving the Dio1 adenovirus after 12 days as shown in FIG. 7. To generate adenoviral recombinants, recombinant adenovirus Ad-Dio1 was made using the AdEasy system. First, the mouse deiodinase 1 gene was cloned into a shuttle vector pAdTrack-CMV (Stratagene) using XhoI and SalI (New England BioLabs). Second, the linearized recombinant construct was transformed together with a supercoiled adenoviral vector pAdEasy-1 (Stratagene) into E. coli strain BJ5183 (Stratagene). Third, the recombinant adenoviral construct was cleaved with PacI and transfected into a 293 cell line. Virus stocks were amplified in HEK293 cells on 15 cm plates and purified using Vivapure AdenoPACK 100 Adenovirus Purification Kit (Sartorius Biotech). A control vector (Adv/GFP) carrying cDNA for green fluorescence protein was also prepared as described above. Sixteen week old LIRKO mice were injected intravenously with Ad-Dio1 or Ad-GFP (as a control). Blood and livers from these two groups were collected on day 12 after injection. Total RNA were extracted, then reverse transcription and qPCR were performed as mentioned above (FIG. 4).

The results in FIG. 8 show that an increased expression of Dio1 was associated with a 25% increase in the expression of ApoApoA-1 mRNA, which trended toward significance. The experiment was done as for FIG. 7. Moreover, HDL cholesterol levels, assessed by FPLC, increased in each of the mice injected with the Dio1 adenovirus. FIG. 9. 200 μl samples of serum from individual LIRKO mice treated with Ad-Dio1 and Ad-GFP on day 12 after virus injection were subjected to FPLC. Total cholesterol content in each fraction was determined by enzymatic kit (Wako diagnostic).

Additionally, ApoA-1 in the HDL fractions isolated from the FPLC was also increased in mice receiving the adenovirus. Western blots of FPLC fraction from LIRKO mice treated with Ad-Dio1 and Ad-GFP, mice injected with Ad-Dio1 and Ad-Dio1 (depicted as Ad-5 and Ad-6) each had a higher ApoA-1 level in HDL (fractions 29-32) than the two mice injected with Ad-GFP and Ad-GFP. Samples from FPLC fractions containing ApoA-1 were run individually on 12% SDS PAGE. Data not shown.

In the subsequent procedures, 0.5 μl of serum from 4 h fasted 4 month old LIRKO and Floxed mice were run on SDS-polyacrylamide gel electrophoresis, Protein bands on the gel were transferred to nitrocellulose membrane (Bio Rad). Incubation of anti-mouse ApoA-1 antibody (Calbiochem) with the membrane was performed in TBST including 0.1% Tween 20 and 2% nonfat milk at 4° C. overnight. Detection of the immune complexes was carried out by ECL Western Blotting Substrate (Thermo Scientific).

FIG. 10 shows that when insulin receptors in McArdle RH7777 hepatoma cells were knocked down with a siRNA, the expression of both Dio1 mRNA and ApoA-1 mRNA were also significantly reduced. For RNA interference transfection, McArdle RH7777 rat hepatoma cells (cultured with DMEM, 10% FBS, 10% horse serum, 1% p/s) were seeded in six-well plates at 2.5×10⁵ cells/well. A pool (40 nM final concentration) of two individual Mission siRNA oligonucleotides (SASI_Rn02_{—}00261274, siRNA1, M SASI_Rn02_{—}00261275, siRNA2, Sigma-Aldrich) was transfected into McArdle RH7777 (McA) cells using nanoparticle-based siRNA transfection reagent (N-TER™, Sigma-Aldrich) 16 h later. Mission siRNA Universal Negative Control (#1; Sigma-Aldrich) was used as a negative control. Cells were collected at 36-48 h after transfection. Total RNA were extracted, then reverse transcription and qPCR were performed as mentioned above (FIG. 6).

Primers (forward, reverse) used for this study were as follows:

Rat (r)-Insulin receptor,
(SEQ ID NO: 17)
ATGGGCTTCGGGAGAGGAT,
(SEQ ID NO: 18)
GGATGTCCATACCAGGGCAC;
rDeiodinase 1,
(SEQ ID NO: 19)
CCCCTGGTGTTGAACTTTG,
(SEQ ID NO: 20)
TGTGGCGTGAGCTTCTTC;
rapoA-1,
(SEQ ID NO: 21)
CCTGGATGAATTCCAGGAGA,
(SEQ ID NO: 22)
TCGCTGTAGAGCCCAAACTT.

### Example 5

### Dio1 Increases ApoA-1 Promotor Activity

As shown in FIG. 11, when Dio1 was knocked down directly in McArdle RH7777 cells with a siRNA, the activity of a rat ApoA-1 promoter was reduced, as assessed by a luciferase reporter assay. These data indicate a direct effect of Dio1 on transcription of the ApoA-1 gene.

PGL3-luciferase reporter plasmid contains the ApoA-1 promoter sequence from base pair −256 to +1 upstream of the luciferase gene (PGL3-ApoA-1-LUC) (a gift from Dr. Bart Staels). Mission siRNA for deiodinase 1 (SASI_Rn01_{—}00118856 siRNA1, siRNA 1, SASI_Rn01_{—}00118857 siRNA 2) was obtained from Sigma-Aldrich. McArdle RH7777 rat hepatoma cells were seeded in 24 well plates at 5×10⁴ cells/well 16 h before transfection. A pool of two siRNA1&2 (80 nM final concentration) was co-transfected with plasmid PGL3-Apo-AI-LUC (200 ng/well) and control PGL3-LUC (200 ng/well) using 1.50 μl/well Lipofectamine™ 2000 (Invitrogen) in serum-free medium. After 6 hours of transfection, the transfection medium was replaced with culture medium containing 10% FBS and 10% horse serum. At 48 h after transfection, the cells were washed twice in ice-cold PBS and lysed with reporter lysis buffer (luciferase assay kit, Promega) on ice for 20 minutes. The cells were then scraped down and spun at 14,000 rpm for 10 minutes in cold room. The supernatant was collected for luciferase activity assay.

As shown in FIG. 12, ApoA-1 mRNA levels were significantly reduced in livers from Dio1 knockout mice (wt: 8, Dio1KO: 7). Total RNA was isolated from the mouse liver using TRIzol reagent (Invitrogen), mRNA levels were quantified by quantitative PCR with SYBR Green (Agilent Technologies). cDNA was synthesized from 4 μg of total RNA by using SuperScript II reverse transcriptase (Invitrogen).

### Example 6

### Region B of the ApoA-1 Promoter Is the Target For Insulin Signaling

FIG. 17A is a schematic representation of human ApoA-1 promoter constructs that were used to identify the region of the promoter that is responsive to insulin. These constructs are described in Claudel. et al. JCI, 2002. Constructs A and C contain HREs (hormone response elements) which bind nuclear receptor superfamily such as PPARalpha, thyroid receptors, estrogen receptors, retinoid receptors and Construct B binds other types of transcription factors such as those in the C/EBP family. However, the possibility for additional factors binding to these regions is high.

Experiments were conducted in which HepG2 cells were transfected with ApoA-1-Luc genes that comprised all three regions—ABC, only regions B and C or only region C. These regions have been previously mapped and have distinct complements of response elements. 6 hrs later, the cells were infected with ad-sh-Insr or Ad-sh-GFP. The results showed that an insulin or Dio1 responsive region located somewhere in the B construct was required for the effects of insulin signaling on promoter activity. In other words, insulin increases human ApoA-1 promoter activity by affecting a region located in the B Construct portion of the promoter that comprises −192 to −128bp, relative to the transcription start site. Without being bound by theory, it is almost certain that neither insulin nor Dio1 bind to the ApoA-I promoter. Instead, they cause generation of some other molecule that binds to the promoter.

Thus certain embodiments of the screening methods described below preferably use the B construct of the ApoA-1 promoter as a target, and test agents that increase ApoA-1 promoter activity are further tested to determine that the agents also increase ApoA-1 expression.

### Example 7

### Screening For Agents That Increase Expression Or Biological Activity of Dio1 Or ApoA-1 Through Their Respective Promoters

Based on preliminary data, molecules that increase expression of Dio1 will likely also increase expression of ApoA-1; however, small molecules that increase ApoA-1 will not affect Dio1 gene expression. The test agents of interest clinically are primarily those that will increase ApoA-1 expression via increasing Dio1 expression. Using transfected cells that also have reduced insulin receptors will increase the specificity of any positive screens for ApoA-1 gene expression, and without being bound by theory, it is likely that these molecules will work by increasing Dio1 expression. The screening assays described look for agents that act on Dio1 or ApoA-1 promoters.

Embodiments of methods for a high-throughput screening assay for compounds that increase the expression of Dio1 or ApoA-1 through their respective promoters, involve first creating stable cell lines, for example a human hepatoma cell line HepG2 cells or rat hepatoma cell line McARH7777 cells, that have been transfected with a nucleic acid encoding the Dio1 or ApoA-1 promoter (or biologically active fragment of the promoter such as Construct B for the ApoA-1 promoter) operatively linked to a reporter protein, preferably one that can be visualized. In an example, a cDNA construct will be made containing the human promoter linked to a reporter construct such as a luciferase or a GFP reporter construct; such reporter constructs are well known to persons of skill in the art. Any reporter construct known in the art can be used in these screening assays if it permits easy and rapid detection. In some embodiments of the screens, test agents are screened for their ability to increase the activity of the targeted Dio1 or ApoA-1 promoters (especially the Construct B of ApoA-1 promoter), and then are screened to confirm/determine if the test agent is also able to increase expression of the respective protein either by assaying increases in mRNA expression or by assaying Dio1 or ApoA-1 mRNA or protein levels using methods known in the art, such as PCR or ELISAs

In an embodiment, the transfected cells will be grown in 96-well plates that will be assayed to determine the effect of test compounds on the expression of the Dio1 promoter by determining the amount of reporter protein (for example a fluorescent product such as luciferase or GFP) in treated versus untreated cells. In another embodiment the sensitivity of this assay is increased by using transfected cells that have been contacted with an inhibitory oligonucleotide such as antisense RNA, siRNA or shRNA or using cells in which the insulin receptor gene has been knocked out. In yet another embodiment, based on the results described in Example 6 showing that Construct B of the ApoA-1 promoter has a response element affected by insulin signaling, the target promoter is Construct B of the apoA-1 promoter. Test agents that increase activity of the targeted promoter are preferably then tested to confirm/determine that they increase expression of the respective protein. The reporter can be a fluorophore, such as Fluorescein isothiocyanate (FITC), Phycoerythrin (PE), R Phycoerythrin-Cyanin 5.1 (PC5), allophycocyanine (APC), PerCP, and others well known in the art. Any label that can be detected and quantified can be used, such as alkaline phosphatase, horseradish peroxidase, urease, beta galactosidase, and chloramphenicol acyltransferase.

In some embodiments for testing agents that affect Dio1 or ApoA-1 promoter activity, cells are used in which insulin receptors have been knocked down or knocked out, for example, it was shown above that siRNA targeting insulin receptors in McARH7777 cells resulted in reduced expression of a rat ApoA-1-luciferase reporter construct.

See Table 1 for nucleic acid and amino acid sequences for Dio1 protein, Dio1 gene, ApoA-1 protein; ApoA-1 gene and insulin receptor protein and gene. Any mammalian cell that can express the nucleic acid constructs described herein, and allow the promoter and/or gene product to function can be used. Preferred cells are hepatoma cells, such as human hepatoma cell line HepG2 or rat hepatoma cell line McARH7777. The “nucleic acid targets” in the assays are the Dio1 promoter and/or the ApoA-1 promoter (preferably Construct B).

The term “test agent” as used herein includes any molecule, e.g., protein, oligopeptide, small organic molecule, peptidomimetics, antibodies, polysaccharide, polynucleotide, lipid, etc., or mixtures thereof, for use in embodiments of the screening methods that evaluate the ability of a “test agent” to directly or indirectly alter the expression or bioactivity of a “targeted protein” which is either Dio1 or ApoA-1. The methods are designed to identify agents that modulate (increase or reduce) the activity of a nucleic acid target, thereby increasing the expression of a targeted protein. Generally a plurality of assay mixtures is run in parallel with different agent concentrations to obtain a differential response to the various concentrations. Typically, one of these concentrations serves as a negative control, i.e., at zero concentration or below the level of detection. It is to be noted that the compositions of the invention include pharmaceutical compositions comprising one or more of the agents identified via the herein described screening methods. Such pharmaceutical compositions can be formulated, for example, as discussed, below.

Test agents may include, but are not limited to, peptides such as, for example, soluble peptides, including but not limited to members of random peptide libraries (see, e.g. Lam, K. S. et al., 1991, Nature 354:82-84; Houghten, R. et al., 1991, Nature 354:84-86); and combinatorial chemistry-derived molecular library made of D- and/or L-configuration amino acids, phosphopeptides (including, but not limited to, members of random or partially degenerate, directed phosphopeptide libraries; (see, e.g., Songyang, Z. et al., 1993, Cell 72:767-778), antibodies (including, but not limited to, polyclonal, monoclonal, humanized, anti-idiotypic, chimeric or single chain antibodies, and FAb, F(ab′).sub.2 and FAb expression library fragments, and epitope binding fragments thereof), and small organic or inorganic molecules.

Known and novel pharmacological agents identified in screens may be further subjected to directed or random chemical modifications, such as acylation, alkylation, esterification, amidification to produce structural analogs. The agent may be a protein. By “protein” in this context is meant at least two covalently attached amino acids, which includes proteins, polypeptides, oligopeptides and peptides. The protein may be made up of naturally occurring amino acids and peptide bounds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acids” also includes imino acid residues such as proline and hydroxyproline. The side chains may be in either the (R) or the (S) configuration. In the preferred embodiment, the amino acids are in the (S) or L-configuration. If non-naturally occurring side chains are used, non-amino acid substituents may be used, for example to prevent or retard in vivo degradations

The agent may be a naturally occurring protein or fragment or variant of a naturally occurring protein. Thus, for example, cellular extracts containing proteins, or random or directed digests of proteinaceous cellular extracts, may be used. In this way, libraries of prokaryotic and eukaryotic proteins may be made for screening against one of the various proteins. Libraries of bacterial, fungal, viral, and mammalian proteins, with the latter being preferred, and human proteins being especially preferred may be used. Agents may be peptides of from about 5 to about 30 amino acids, with from about 5 to about 20 amino acids being preferred, and from about 7 to about 15 being particularly preferred. The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized agent bioactive proteinaceous agents. Further variations and details are set forth in Karsenty US application 20100190697.

As used herein, the term “nucleic acid” refers to both RNA and DNA, including cDNA, genomic DNA, and synthetic (e.g., chemically synthesized) DNA. The nucleic acid can be double-stranded or single-stranded (i.e., a sense or an antisense single strand).

### Gene Therapy

In some embodiments the Dio1 gene encoding Dio1 or a biologically active fragment or variant thereof, is introduced to a cell in a subject having chronically low plasma HDLC, preferably into a liver cell, to achieve intracellular concentrations of Dio1 that activate the ApoA-1 promoter thereby increasing ApoA-1 expression that in turn increases HDLC. Therefore a recombinant DNA construct in which expression of the therapeutic nucleic acid molecule is placed under the control of a promoter can be used for gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62:191-217; May, 1993, TIBTECH 11(5):155-215).

In the present embodiments, a gene encoding Dio1 including biologically active fragments or variants thereof, may be administered as a therapy to increase plasma HDLC in a subject. Methods commonly known in the art of recombinant DNA technology that can be used in embodiments of the invention are described in Ausubel et al. (eds.), 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds.), 1994, Current Protocols in Human Genetics, John Wiley & Sons, NY.

### Pharmaceutical Formulations

As used herein, a “therapeutically effective amount” of Dio1 is an amount sufficient to increase blood (plasma) levels of HDLC and/or ApoA-1 in a subject.

Certain embodiments of the present invention are directed to pharmaceutical compositions and formulations comprising deiodinase 1 or biologically active fragments or variants thereof, preferably human Dio1, in an amount that increases plasma HDLC, preferably formulated to target the liver. Certain experiments show that Dio1 increases ApoA-1, which is a valuable clinical tool for treating in a subject having with suppressed insulin signaling. In a preferred embodiment, such formulations may be formulated in liposomes and lipid nanoparticles that targeted to the liver. Liposomes are cleared from the circulation by macrophages of the RES, in particular those of the liver and spleen (Gregoriadis, 1976; Weinstein, 1984; Senior, 1987). Certain embodiments of the present invention are directed to pharmaceutical compositions and formulations comprising a 2-Acetamido-2-deoxy-D-galactose (Ga1NAc) conjugate of Dio1 or a biologically active protein or variant that has at least 70% identity with the amino acid sequence of Dio1 with, for hepatocyte specific delivery via asialoglycoprotein receptor. (Akinc, Formulation and Delivery of Peptides and Oligonucleotides, Strategies for Delivery of RNAi Therapeutics, AsiaTIDES 2012, Toyko, Japan, Mar. 2, 2012). The hepatocyte targeted delivery of macromolecular drugs was demonstrated by Li et al via asialoglycoprotein receptor (ASGPR) (Li, et al., Targeted delivery of macromolecular drugs, asialoglycoprotein receptor (ASGPR) expression by selected hepatoma cell lines used in antiviral drug development, Curr Drug Deliv. 5 (4) October 2008, pp. 299-302). Therefore other embodiments are directed to a Ga1NAc conjugate of Dio1 for delivery via asialoglycoprotein receptor (ASGPR).

The therapeutic agents are generally administered preferably intravenously or subcutaneously in a therapeutically effective amount sufficient raise blood HDLC to a desired level. However, routine experimentation may reveal other effective routes, therefore the term “administer” is used in its broadest sense and includes any method of introducing the compositions of the present invention into a subject that achieves the desired result. There are adenoviral vectors called AAV (adeno-associated virus) that give long term expression with minimal to no inflammatory response. They are being used in gene therapy and are typically given IV. AAV go almost exclusively to the liver and that is needed to raise ApoA-1 production as almost all ApoA-1 is made in the liver. Recombinant proteins have been given IV or SC. For example, EPOGEN® (epoetin alfa), which is used to treat a lower than normal number of red blood cells (anemia) caused by chronic kidney disease in patients on, is given subcutaneously.

The pharmaceutical compositions of the present invention may be administered in a number of ways depending upon whether local or systemic treatment is desired

Administration can be oral, intravenous, parenteral/intra-arterial, subcutaneous, intraperitoneal or intramuscular injection or infusion. The term “slow release” refers to the release of a drug from a polymeric drug delivery system over a period of time that is more than one day wherein the active agent is formulated in a polymeric drug delivery system that releases effective concentrations of the drug.

Therapeutic Dio1 can be administered as a single treatment or, preferably, can include a series of treatments, that continue at a frequency and for a duration of time that achieves the desired effect.

The appropriate dose of an active agent depends upon a number of factors within the ken of the ordinarily skilled physician, veterinarian, or researcher. The dose(s) vary, for example, depending upon the identity, size, and condition of the subject or sample being treated, further depending upon the route by which the composition is to be administered, and the effect which the practitioner desires the an active agent to have. It is furthermore understood that appropriate doses of an active agent depend upon the potency with respect to the expression or activity to be modulated. Such appropriate doses may be determined by monitoring plasma ApoAl or HDLC levels, for example. it is understood that the specific dose level for any particular subject will depend upon a variety of factors including the activity of the specific compound employed, the age, body weight, general health, gender, and diet of the subject, the time of administration, the route of administration, the rate of excretion, any drug combination, and the degree of expression or activity to be modulated.

The Dio1 can be formulated with an acceptable carrier using methods well known in the art. The actual amount of therapeutic agent will necessarily vary according to the particular formulation, route of administration, and dosage of the pharmaceutical composition, the specific nature of the condition to be treated, and possibly the individual subject. The dosage for the pharmaceutical compositions of the present invention can range broadly depending upon the desired effects, the therapeutic indication, and the route of administration, regime, and purity and activity of the composition.

A suitable subject can be an individual or animal that is has lower than desired HDLC levels or ApoA-1.

Techniques for formulation and administration can be found in “Remington: The Science and Practice of Pharmacy” (20th edition, Gennaro (ed.) and Gennaro, Lippincott, Williams & Wilkins, 2000.

Active agents (Dio1) may be admixed, encapsulated, conjugated or otherwise associated with other molecules, molecule structures or mixtures of compounds, as for example, liposomes, receptor targeted molecules, or other formulations, for assisting in uptake, distribution and/or absorption. Representative United States patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

### Protein Variants

Dio1 for therapeutic use that falls within the scope of the invention include biologically active fragments and variants that are substantially homologous to human Dio1 including proteins derived from another organism, i.e., an ortholog and isoforms of Dio1. As used herein, two proteins are substantially homologous, or identical, when their amino acid sequences are at least about 70-75%, typically at least about 80-85%, and most typically at least about 90-95%, 97%, 98% or 99% or more homologous. The homologous proteins can be described by their % identity/homology. “Homology” or % identity between two amino acid sequences or nucleic acid sequences can be determined by using the algorithms disclosed herein. These algorithms can be used to determine percent identity between two amino acid sequences or nucleic acid sequences.

To determine the percent homology or percent identity of two amino acid sequences or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). Preferably, the length of a reference sequence aligned for comparison purposes is at least 70%, 80%, or 90% or more of the length of the sequence that the reference sequence is compared to. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The invention also encompasses polypeptides with less than 70% identity that have sufficient similarity so as to perform one or more of the same functions performed by DIO. Similarity is determined by considering conserved amino acid substitutions. Such substitutions are those that substitute a given amino acid in a polypeptide by another amino acid of like characteristics. Conservative substitutions are likely to be phenotypically silent. Guidance concerning which amino acid changes are likely to be phenotypically silent is found in Bowie et al., Science 247:1306-1310 (1990). Variants include conservative Amino Acid Substitutions: Aromatic Phenylalanine Tryptophan Tyrosine Hydrophobic Leucine Isoleucine Valine Polar Glutamine Asparagine Basic Arginine Lysine Histidine Acidic Aspartic Acid Glutamic Acid Small Alanine Serine Threonine Methionine Glycine.

Examples of conservative substitutions are the replacements, one for another, among the hydrophobic amino acids Ala, Val, Leu, and Ile; interchange of the hydroxyl residues Ser and Thr; exchange of the acidic residues Asp and Glu; substitution between the amide residues Asn and Gln; exchange of the basic residues Lys, His and Arg; replacements among the aromatic residues Phe, Trp and Tyr; exchange of the polar residues Gln and Asn; and exchange of the small residues Ala, Ser, Thr, Met, and Gly.

The comparison of sequences and determination of percent identity and homology between two polypeptides can be accomplished using a mathematical algorithm. For example, Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H G., eds., Humana Press, New Jersey, 1994; Sequence Analysis in Molecular Biology, van Heinje, G., Academic Press, 1987; and Sequence Analysis Primer, Gribskov, M. and Devereux, J., eds., M Stockton Press, New York, 1991. A non-limiting example of such a mathematical algorithm is described in Karlin et al. (1993) Proc. Natl. Acad. Sci. USA 90:5873-5877. The percent identity or homology between two amino acid sequences may be determined using the Needleman et al. (1970) (.I Mol. Biol. 48:444-453) algorithm. Another non-limiting example of a mathematical algorithm that may be utilized for the comparison of sequences is the algorithm of Myers and Miller, CABIOS (1989).

A substantially homologous protein, according to the present invention, may also be a polypeptide encoded by a nucleic acid sequence capable of hybridizing to a sequence having at least 70-75%, typically at least about 80-85%, and most typically at least about 90-95%, 97%, 98% or 99% identity to the targeted nucleic acid sequence, under stringent conditions, e.g., hybridization to filter-bound DNA in 0.5 M NaHPO.sub.4, 7% sodium dodecyl sulfate (SDS), 1 mM EDTA at 65.degree. C., and washing in 0.1.times.SSC/0.1% SDS at 68.degree. C. (Ausubel F.M. et al., eds., 1989, Current Protocols in Molecular Biology, Vol. I, Green Publishing Associates, Inc., and John Wiley & sons, Inc., New York, at p. 2.10.3) and encoding a functionally equivalent gene product; or under less stringent conditions, such as moderately stringent conditions, e.g., washing in 0.2.times.SSC/0.1% SDS at 42.degree. C. (Ausubel et al., 1989 supra), yet which still encodes a biologically active protein or fragment.

Peptides corresponding to fusion proteins are also within the scope of the invention and can be designed on the basis of the Dio1 nucleotide and amino acid sequences disclosed herein using routine methods known in the art. The Gene Bank Nos for genes, mRNA and proteins used in the various embodiments are set forth in Table 1.

TABLE 1

| Gene | mRNA | Protein |
|---|---|---|
| hDio1isoform a | NM_000792 | NP_000783 |
| hDio1isoform c | NM_001039715 | NP_001034804 |
| hDio1isoform b | NM_001039716 | NP_001034805 |
| hDio1isoform d | NM_213593 | NP_998758 |
| hINSR long isoform | NM_000208 | NP_000199 |
| preproprotein | | |
| hINSR short iosform | NM_001079817 | NP_001073285 |
| preproprotein | | |
| hApoA-1 | NM_000039 | NP_000030 |
| Mouse ApoA-1 | | NP_033822 |
| Rat ApoA-1 | | NP_036870 |

In an embodiment of the invention, the Dio1 is fused to a polypeptide capable of targeting the Dio1 to the liver. A fusion protein can also be made as part of a chimeric protein for drug screening or use in making recombinant protein. These comprise a peptide sequence operatively linked to a heterologous peptide. “Operatively linked” in this context indicates that the peptide and the heterologous peptide are fused in-frame. The heterologous peptide can be fused to the N-terminus or C-terminus of the target peptide or can be internally located. In one embodiment, the fusion protein does not affect the function of the peptide (such as Dio1) function. For example, the fusion protein can be a GST-fusion protein. Other types of fusion proteins include, but are not limited to, enzymatic fusion proteins, for example beta-galactosidase fusions, yeast two-hybrid GAL-4 fusions, poly-His fusions and Ig fusions. Such fusion proteins, particularly poly-His fusions, can facilitate the purification of recombinant Dio1. In certain host cells (e.g., mammalian host cells), expression and/or secretion of a protein can be increased by using a heterologous signal sequence. EP-A 0 464 533

Polypeptides often contain amino acids other than the 20 amino acids commonly referred to as the 20 naturally-occurring amino acids. Further, many amino acids, including the terminal amino acids, may be modified by natural processes, such as processing and other post-translational modifications, or by chemical modification techniques well known in the art. Common modifications that occur naturally in polypeptides are described below.

Dio1 also encompass derivatives that contain a substituted amino acid residue that is not one encoded by the genetic code, in which a substituent group is included, in which the mature polypeptide is fused with another compound, such as a compound to increase the half-life of the polypeptide (for example, polyethylene glycol), or in which the additional amino acids are fused to the Dio1 polypeptide such as a leader or secretory sequence or a sequence for purification of the Dio1 polypeptide or a pro-protein sequence.

A protein can be modified according to known methods in medicinal chemistry to increase its stability, half-life, uptake or efficacy. Known modifications include, but are not limited to, acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cros slinks, formation of cystine, formation of pyroglutamate, formylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination.

Several particularly common modifications that may be used, such as glycosylation, lipid attachment, sulfation, hydroxylation and ADP-ribosylation are described in most basic texts, such as Proteins—Structure and Molecular Properties, 2nd ed., T. E. Creighton, W. H. Freeman and Company, New York (1993). Many detailed reviews are available on this subject, such as by Wold, F., Posttranslational Covalent Modification of Proteins, B. C. Johnson, Ed., Academic Press, New York 1-12 (1983); Seifter et al. (1990) Meth. Enzymol. 182: 626-646) and Rattan et al. (1992) Ann. NY: Acad. Sci. 663:48-62.

Modifications can occur anywhere in the protein and its fragments and variants, including the peptide backbone, the amino acid side-chains and the amino or carboxyl termini. Recombinant or isolated Dio1 and its fragments and variants with N-formylmethionine as the amino terminal residue are within the scope of the present invention. A brief description of various protein modifications that come within the scope of this invention are described in Karsenty, US Application 20100190697.

Some common modifications are set forth below:

| Protein Modification | Description |
|---|---|
| Acetylation | Acetylation of N-terminus or e-lysines. Introducing an acetyl |
| | group into a protein, specifically, the substitution of an acetyl |
| | group for an active hydrogen atom. |
| | A reaction involving the replacement of the hydrogen atom of a |
| | hydroxyl group with an acetyl group (CH₃CO) yields a specific |
| | ester, the acetate. Acetic anhydride is commonly used as an |
| | acetylating agent, which reacts with free hydroxyl groups. |
| | Acylation may facilitate addition of other functional groups. A |
| | common reaction is acylation of e.g., conserved lysine residues |
| | with a biotin appendage. |
| ADP-ribosylation | Covalently linking proteins or other compounds via an arginine- |
| | specific reaction. |
| Alkylation | Alkylation is the transfer of an alkyl group from one molecule to |
| | another. The alkyl group may be transferred as an alkyl |
| | carbocation, a free radical or a carbanion (or their equivalents). |
| | Alkylation is accomplished by using certain functional groups |
| | such as alkyl electrophiles, alkyl nucleophiles or sometimes |
| | alkyl radicals or carbene acceptors. A common example is |
| | methylation (usually at a lysine or arginine residue). |
| Amidation | Reductive animation of the N-terminus. Methods for amidation |
| | of insulin are described in U.S. Pat. No. 4,489,159. |
| Carbamylation | Nigen et al. describes a method of carbamylating hemoglobin. |
| Carboxylation | Carboxylation typically occurs at the glutamate residues of a |
| | protein, which may be catalyzed by a carboxylase enzyme (in |
| | the presence of Vitamin K—a cofactor). |
| Citrullination | Citrullination involves the addition of citrulline amino acids to |
| | the arginine residues of a protein, which is catalyzed by |
| | peptidylarginine deaminase enzymes (PADs). This generally |
| | converts a positively charged arginine into a neutral citrulline |
| | residue, which may affect the hydrophobicity of the protein (and |
| | can lead to unfolding). |
| Condensation of amines | Such reactions, may be used, e.g., to attach a peptide to other |
| with aspartate or glutamate | proteins labels. |
| Covalent attachment of flavin | Flavin mononucleotide (FAD) may be covalently attached to |
| | serine and/or threonine residues. May be used, e.g., as a light- |
| | activated tag. |
| Covalent attachment of | A heme moiety is generally a prosthetic group that consists of an |
| heme moiety | iron atom contained in the center of a large heterocyclic organic |
| | ring, which is referred to as a porphyrin. The heme moiety may |
| | be used, e.g., as a tag for the peptide. |
| Attachment of a nucleotide | May be used as a tag or as a basis for further derivatising a |
| or nucleotide derivative | peptide. |
| Cross-linking | Cross-linking is a method of covalently joining two proteins. |
| | Cross-linkers contain reactive ends to specific functional |
| | groups (primary amines, sulfhydryls, etc.) on proteins or other |
| | molecules. Several chemical groups may be targets for |
| | reactions in proteins and peptides. For example, Ethylene |
| | glycol bis[succinimidylsuccinate, Bis[2- |
| | (succinimidooxycarbonyloxy)ethyl] sulfone, and |
| | Bis[sulfosuccinimidyl] suberate link amines to amines. |
| Cyclization | For example, cyclization of amino acids to create optimized |
| | delivery forms that are resistant to, e.g., aminopeptidases (e.g., |
| | formation of pyroglutamate, a cyclized form of glutamic acid). |
| Disulfide bond formation | Disulfide bonds in proteins are formed by thiol-disulfide |
| | exchange reactions, particularly between cysteine residues (e.g., |
| | formation of cystine). |
| Demethylation | See, e.g., U.S. Pat. No. 4,250,088 (Process for demethylating lignin). |
| Formylation | The addition of a formyl group to, e.g., the N-terminus of a |
| | protein. See, e.g., U.S. Pat. Nos. 4,059,589, 4,801,742, and 6,350,902. |
| Glycylation | The covalent linkage of one to more than 40 glycine residues to |
| | the tubulin C-terminal tail. |
| Glycosylation | Glycosylation may be used to add saccharides (or |
| | polysaccharides) to the hydroxy oxygen atoms of serine and |
| | threonine side chains (which is also known as O-linked |
| | Glycosylation). Glycosylation may also be used to add |
| | saccharides (or polysaccharides) to the amide nitrogen of |
| | asparagine side chains (which is also known as N-linked |
| | Glycosylation), e.g., via oligosaccharyl transferase. |
| GPI anchor formation | The addition of glycosylphosphatidylinositol to the C-terminus |
| | of a protein. GPI anchor formation involves the addition of a |
| | hydrophobic phosphatidylinositol group—linked through a |
| | carbohydrate containing linker (e.g., glucosamine and mannose |
| | linked to phosphoryl ethanolamine residue)—to the C-terminal |
| | amino acid of a protein. |
| Hydroxylation | Chemical process that introduces one or more hydroxyl groups |
| | (—OH) into a protein (or radical). Hydroxylation reactions are |
| | typically catalyzed by hydroxylases. Proline is the principal |
| | residue to be hydroxylated in proteins, which occurs at the C^{γ} |
| | atom, forming hydroxyproline (Hyp). In some cases, proline |
| | may be hydroxylated at its C^{β} atom. Lysine may also be |
| | hydroxylated on its C^{δ} atom, forming hydroxylysine (Hyl). |
| | These three reactions are catalyzed by large, multi-subunit |
| | enzymes known as prolyl 4-hydroxylase, prolyl 3-hydroxylase |
| | and lysyl 5-hydroxylase, respectively. These reactions require |
| | iron (as well as molecular oxygen and α-ketoglutarate) to carry |
| | out the oxidation, and use ascorbic acid to return the iron to its |
| | reduced state. |
| Iodination | See, e.g., U.S. Pat. No. 6,303,326 for a disclosure of an enzyme that is |
| | capable of iodinating proteins. U.S. Pat. No. 4,448,764 discloses, e.g., a |
| | reagent that may be used to iodinate proteins. |
| ISGylation | Covalently linking a peptide to the ISG15 (Interferon- |
| | Stimulated Gene 15) protein, for, e.g., modulating immune response. |
| Methylation | Reductive methylation of protein amino acids with |
| | formaldehyde and sodium cyanoborohydride has been shown to |
| | provide up to 25% yield of N-cyanomethyl (—CH₂CN) product. |
| | The addition of metal ions, such as Ni²⁺, which complex with |
| | free cyanide ions, improves reductive methylation yields by |
| | suppressing by-product formation. The N-cyanomethyl group |
| | itself, produced in good yield when cyanide ion replaces |
| | cyanoborohydride, may have some value as a reversible |
| | modifier of amino groups in proteins. (Gidley et al.) |
| | Methylation may occur at the arginine and lysine residues of a |
| | protein, as well as the N- and C-terminus thereof. |
| Myristoylation | Myristoylation involves the covalent attachment of a myristoyl |
| | group (a derivative of myristic acid), via an amide bond, to the |
| | alpha-amino group of an N-terminal glycine residue. This |
| | addition is catalyzed by the N-myristoyltransferase enzyme. |
| Oxidation | Oxidation of cysteines. |
| | Oxidation of N-terminal Serine or Threonine residues |
| | (followed by hydrazine or aminooxy condensations). |
| | Oxidation of glycosylations (followed by hydrazine or |
| | aminooxy condensations). |
| Palmitoylation | Palmitoylation is the attachment of fatty acids, such as palmitic |
| | acid, to cysteine residues of proteins. Palmitoylation increases |
| | the hydrophobicity of a protein. |
| (Poly)glutamylation | Polyglutamylation occurs at the glutamate residues of a protein. |
| | Specifically, the gamma-carboxy group of a glutamate will form |
| | a peptide-like bond with the amino group of a free glutamate |
| | whose alpha-carboxy group may be extended into a |
| | polyglutamate chain. The glutamylation reaction is catalyzed by |
| | a glutamylase enzyme (or removed by a deglutamylase |
| | enzyme). Polyglutamylation has been carried out at the C- |
| | terminus of proteins to add up to about six glutamate residues. |
| | Using such a reaction, Tubulin and other proteins can be |
| | covalently linked to glutamic acid residues. |
| Phosphopantetheinylation | The addition of a 4′-phosphopantetheinyl group. |
| Phosphorylation | A process for phosphorylation of a protein or peptide by |
| | contacting a protein or peptide with phosphoric acid in the |
| | presence of a non-aqueous apolar organic solvent and contacting |
| | the resultant solution with a dehydrating agent is disclosed e.g., |
| | in U.S. Pat. No. 4,534,894. Insulin products are described to be |
| | amenable to this process. See, e.g., U.S. Pat. No. 4,534,894. Typically, |
| | phosphorylation occurs at the serine, threonine, and tyrosine |
| | residues of a protein. |
| Prenylation | Prenylation (or isoprenylation or lipidation) is the addition of |
| | hydrophobic molecules to a protein. Protein prenylation |
| | involves the transfer of either a farnesyl (linear grouping of |
| | three isoprene units) or a geranyl-geranyl moiety to C-terminal |
| | cysteine(s) of the target protein. |
| Proteolytic Processing | Processing, e.g., cleavage of a protein at a peptide bond. |
| Selenoylation | The exchange of, e.g., a sulfur atom in the peptide for selenium, |
| | using a selenium donor, such as selenophosphate. |
| Sulfation | Processes for sulfating hydroxyl moieties, particularly tertiary |
| | amines, are described in, e.g., U.S. Pat. No. 6,452,035. A process for |
| | sulphation of a protein or peptide by contacting the protein or |
| | peptide with sulphuric acid in the presence of a non-aqueous |
| | apolar organic solvent and contacting the resultant solution with |
| | a dehydrating agent is disclosed. Insulin products are described |
| | to be amenable to this process. See, e.g., U.S. Pat. No. 4,534,894. |
| SUMOylation | Covalently linking a peptide a SUMO (small ubiquitin-related |
| | Modifier) protein, for, e.g., stabilizing the peptide. |
| Transglutamination | Covalently linking other protein(s) or chemical groups (e.g., |
| | PEG) via a bridge at glutamine residues |
| tRNA-mediated addition of | For example, the site-specific modification (insertion) of an |
| amino acids (e.g., arginylation) | amino acid analog into a peptide. |

### Recombinant Dio1

To practice the methods of the invention, it may be desirable to recombinantly express the Dio 1. The cDNA sequence and deduced amino acid sequence of human Dio1 is available from Gene Bank as described above. Dio1 nucleotide sequences may be isolated using a variety of different methods known to those skilled in the art. For example, a cDNA library constructed using RNA from a tissue known to express Dio1 can be screened using a labeled Dio1 probe. Alternatively, a genomic library may be screened to derive nucleic acid molecules encoding the Dio1 protein. Further, Dio1 nucleic acid sequences may be derived by performing a polymerase chain reaction (PCR) using two oligonucleotide primers designed on the basis of known Dio1 nucleotide sequences. The template for the reaction may be cDNA obtained by reverse transcription of mRNA prepared from cell lines or tissue known to express Dio1.

While the Dio1 peptides can be chemically synthesized (e.g., see Creighton, 1983, Proteins: Structures and Molecular Principles, W.H. Freeman & Co., N.Y.), large polypeptides derived from Dio1 and the full length Dio1 itself may be advantageously produced by recombinant DNA technology using techniques well known in the art for expressing a nucleic acid. Such methods can be used to construct expression vectors containing the Dio1 nucleotide sequences and appropriate transcriptional and translational control signals. These methods include, for example, in vitro recombinant DNA techniques, synthetic techniques, and in vivo genetic recombination. (See, for example, the techniques described in Sambrook et al., 1989, supra, and Ausubel et al., 1989, supra).

In the present specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. The contents of all references, pending patent applications and published patents, cited throughout this application are hereby expressly incorporated by reference as if set forth herein in their entirety, except where terminology is not consistent with the definitions herein. Although specific terms are employed, they are used as in the art unless otherwise indicated.

### Human ApoA-I Promoter

Construct A
SEQ ID NO: 23
ccacccg ggagacctgc aagcctgcag acactcccct cccgccccca
ctgaaccctt gacccctg;
base sequences −256 bp to −192 bp
Construct B
SEQ ID NO: 24
CC CTGCAGCCCC CGCAGCTTGC TGTTTGCCCA CTCTATTTGC
CCAGCCCCAGGGACAGAGCT;
base sequences −192 bp to −128 bp
Construct C
SEQ ID NO: 25
gatccttgaa ctcttaagtt ccacattgcc aggaccagtg
agcagcaaca gggccggggc tgggcttatc agcctcccag
cccagaccct ggctgcagac ataaataggc cctgcaagag
ctggctgctt];
base sequences −128 to −41 bp

### Human Dio1 (hDio1) GenBank Reference Number For Nucleic Acid : NM_{—}000792 For Protein: NP_{—}000783

human DIO1 promoter sequence(3.7 kb): GenBank NG_023306,
genomic DNA from 1300 to 5000:
SEQ ID NO: 26
ttcgtcgact tgagttcttg
1321 accgttccag ttttctcttt tttgtcctcc cagcttctct tcctgccaga acttccttct
1381 ccccgacttg cccactcagc cagcccagct tgtgaatggc tgccagattg ctcttctctg
1441 agtacatacc agctcaacca ctttcagcag ctcccctctg catttaggat gaagcccagg
1501 ctcagccttg gattccaggt cctccctggt caggctctag cttttcttct caattctacc
1561 tctgagctcc ccgccacact catttctttc ggacaaactg ttgggccttg tacatctctt
1621 gtactttccc ttgtctttgc ctttgctgac atcggctggt caagaatgcc cttcccctct
1681 ccatcgtcgt ctatatcccc ctcattcatg tgggtccagc tctcctgaca ccttgtcctc
1741 catgaagcca cctcagcttc ctacagctag gcatgtgctc tctcccttcg gctcatggct
1801 ctctgtctgc acctctcctt ggacactgct gcttcctgct cagcacctgg tacctaagca
1861 caagtcttat ttccctgccc agtggagagc ctcaggagag ggtgtgtgtc tgatttatct
1921 ctggattcct cagcatgctc ggcccagggc ctagatgcag caggtagaga aggcacctga
1981 ggcagttggt ttattccgtg tttttcttgt ttttcttttt ctcttttttt tttttttttt
2041 ttttttttga gacagagtct cactctactg cccacgctgg agtgtagggg tatgatcacg
2101 actcactgca tcctcgattt accaggctca agccatcctc ccaccttagc ctccttagta
2161 cctgggacaa caagtgcaca ccacaatgct cggctaattt ttgcattttt tgtagaggtg
2221 gggtttcacc atgttgccca ggctggtctc gaactcctgg actcaagtga tccacccact
2281 tcggccttcc aaaatgctgg gattacaggc atgagccact gtgcctggcc tatcctgtgt
2341 ttttgaaaga atgttcttta gaacctaagt tccacagata tgctttacta tgtagtgttg
2401 cctggtcaaa gtagttggga aaccctgaat actatatccc cctcctatgc aatttcatgt
2461 gcaatttcat gtgcacatga gtgtatgcac atgaggagtt tacagttcca tagaacagat
2521 ggaggtaata aacaaatcct tacagtccta tgaaatgggg gaggctataa aaaaatagaa
2581 cttttgcctg gaggacttgg aagttttcct ggaggaggtg gctctggaac taggtcttga
2641 agaatgagtc agatttttgt agcctgacaa ggaaaaaggg aagagtgttt tagaggggaa
2701 ggcaggagct tcttttgttt tgctgttcat tcataatttt aaaccacagt gcacaaatga
2761 cctcagttta ttcaacaaat gttcactaat tccattggta gtaagagcaa tggtaataac
2821 taacttacca catgcccatg tgccaagcac tgtaacagaa ccaggccaat ttgctgaatg
2881 ccagtcatct gcagttcagt tccctgaaag ccagcttgcc tcatggccaa ttcatggaat
2941 gtacttgcat catgtaactg tccactttca gtgaggcagt ttacatttta aagactgttg
3001 aatttggtct gagccccgtg gctcacgcct ttaattccag cactttggga ggctgaggcg
3061 ggcagatcac ttgaggtcag aagttcaaga ccagcctggc caacatgatg aaacctcgtc
3121 tctactaaaa atacaaaaat cagccaggca tggtggcatg cacctataat cccagctact
3181 cagaaggctg aggcatgaga atcacttgaa cccaggaggc agaggttgca gtgagccgag
3241 atcgcaccac tgcactccag cctgggtgac acagcgagac tctgtcttaa aataaaataa
3301 aataaaatat aaaataaaat aaaaactgtt cagtttgtct ctgctccctg ctgctgcagc
3361 tgagactgaa aattggtagg agtgaccagt tgcagtggcc catgcctgta atcccagcac
3421 tgtgagcggc tgagtgggag gattgcttga acccaggagt tcaagaacag cctgtgcaac
3481 agagtggaac cctgtctcta caaaatattt aaaaattagt gggatgaggt ggtgtgagcc
3541 tgtagtccca gctactcagg aggctgagtt gggggggtca cttaagccca ggaggtcgag
3601 gcttcagtga gccatgttca tgctagtgca ctccagccta ggtaacagag ttaagacctt
3661 gtctcaaaaa taaataagta aataaaatta aaaattttta atggtaagag gaggggactg
3721 aagcaaaaga aaaatctatt tgcaaaatag agtttacttt cagcacatta acccaaagtc
3781 ccctgaaatc ataggtacta acaatacgga aataaacacc atgggcctct gccctggaag
3841 gcctcataac tcagagtgag agatggtgtc gtgacaggga agcagagggc actgggggca
3901 ggaaccctgt taagagtagg gtaaggaggt ggccaaggga aagcttcctg gaggagagga
3961 tggtgtgctg attgtctagg gacagtgaaa ccttggggtg ggtgaggaag aggggaatgg
4021 aaagcagggc agggcacaga ggaggagcag cagaggtctg agatgtggag aagcaacatt
4081 cagtttggca caagtggggt cccagaggca ggaaggggtg aaggatgagg ctgaaggcat
4141 catcaggaac cagagcttac ggggccttgt gtgtcgtagc tgcaggttga ctttatcctg
4201 agagtactgg tgagttctgg aagggtttcc aagagagaag taaacatgat cagttctgct
4261 tattagaaag acattggccg agcatggtgg ctcacacctg taatcccagc actttgggag
4321 gccgaggcca gcgggtcact tgatgtcagg agttcgagac cagcctggcc aacctgatga
4381 aatcctgtct ctactaaaaa tacaaaaatt agccgggcat cgtggcatgc gcctgtaatc
4441 ccagctcctt gggaggctga ggcaggagaa ttgcttgaac ccgggaggtg gagtttgtag
4501 tgagctgaga ttgcgccact gcactccagc ctgggcaaca aagcgagact ctgtctcaaa
4561 aaaaaaaaaa aaaaaagaga catgttgtaa ctactttgga aacccaccag gccaccaaaa
4621 agctctgttg tatgctttgg gtataaactc tgaactcaga gccagagaca gagagacgtg
4681 aagaatcttt actgataatc taaagcaacc gcttcgtttt tgagatgcaa aagtccagag
4741 aggtgaatga ctcgcttaga gtcacacagt gagttcttag aagagccaga actagacttc
4801 tgactctcag ctcgtgcact tgctgctact ggatacgaca gcaggagctc agggaaactc
4861 tcagccacct ccagccctct gtgcgtccac acacgcacac acacacaata tacacacact
4921 cttggacaca cacagaacaa aacatcgagt aactggcatg gtgtggcaga aggcaagttc
4981 tggatgattt actttctgga

### REFERENCE LIST

The contents of each of the following is hereby incorporated by reference as if fully set forth herein, except for terminology that is inconsistent with the terminology used herein,
1. Gordon, T., Kannel, W. B., Castelli, W. P., and Dawber, T. R. 1981. Lipoproteins, cardiovascular disease, and death. The Framingham Study. Arch Intern Med 141:1128-1131.
2. Gofman, J. W., Young, W., and Tandy, R. 1966. Ischemic heart disease, atherosclerosis, and longevity. Circulation 34:679-697.
3. Miller, G. J., and Miller, N. E. 1975. Plasma high density lipoprotein concentration and development of ischaemic heart disease. Lancet I:16-19.
4. McQueen M J, Hawken S, ang X, and unpuu S, S.A.P.J.S.K.S.J.H.M.V.E.K.K.Y.S. 2008. Lipids, lipoproteins, and apolipoproteins as risk markers of myocardial infarction in 52 countries (the INTERHEART study): a case-control study. 224-233.
5. Reaven, G. M. 1988. Role of insulin resistance in human disease. Diabetes 37:1595-1607.
6. Chahil T. J., and Ginsberg G. N. 2006. Diabetic dyslipidemia. Endocrinol Metab Clin N Am 35:491-510.
7. Tall, A. R. 1990. Plasma high density lipoproteins. Metabolism and relationship to atherogenesis. J Clin Invest 86:379-384.
8. Davis, C. E., Gordon, D., LaRosa, J., Wood, P. D. S., and Halperin, M. 1980. Correlations of plasma high density lipoprotein cholesterol levels with other plasma lipid and lipoprotein concentrations. Circulation 62:IV:24-30.
9. Albrink, M. J., Krauss, R. M., Lindgren, F. T., Von der Groeben, V. D., and Wood, P. D. S. 1980. Intercorrelations among high density lipoproteins, obesity, and triglycerides in a normal population. Lipids 15:668-678.
10. Haas, M. J., Horani, M. H., Wong, N. C., and Mooradian, A. D. 2004. Induction of the apolipoprotein Al promoter by Spl is repressed by saturated fatty acids. Metabolism 53:1342-1348.
11. Lam, J. K., Matsubara, S., Mihara, K., Zheng, X. L., Mooradian, A. D., and Wong, N. C. 2003. Insulin induction of apolipoprotein A1, role of Sp1. Biochemistry 42:2680-2690.
12. Hargrove, G. M., Junco, A., and Wong, N. C. 1999. Hormonal regulation of apolipoprotein A1. J Mol Endocrinol 22:103-111.
13. Biddinger, S. B., Hernandez-Ono, A., Rask-Madsen, C., Haas, J. T., Aleman, J. O., Suzuki, R., Scapa, E. F., Agarwal, C., Carey, M. C., Stephanopoulos, G. et al 2008. Hepatic insulin resistance is sufficient to produce dyslipidemia and susceptibility to atherosclerosis. Cell Metab 7:125-134.
14. Larsen, P. R., and Berry, M. J. 1995. Nutritional and hormonal regulation of thyroid hormone deiodinases. Annu Rev Nutr 15:323-352.
15. Koenig, R. J. 2005. Regulation of type 1 iodothyronine deiodinase in health and disease. Thyroid 15:835-840.
16. Schneider, M. J., Fiering, S. N., Thai, B., Wu, S. Y., St Germain, E., Parlow, A. F., St Germain, D. L., and Galton, V. A. 2006. Targeted disruption of the type 1 selenodeiodinase gene (Dio1MMMM) results in marked changes in thyroid hormone economy in mice. Endocrinology 147:580-589.
17. St Germain, D. L., Galton, V. A., and Hernandez, A. 2009. Minireview: Defining the roles of the iodothyronine deiodinases: current concepts and challenges. Endocrinology 150:1097-1107.
18. Maia, A. L., Goemann, I. M., Meyer, E. L., and Wajner, S. M. 2011. Type 1 iodothyronine deiodinase in human physiology and disease: Deiodinases: the balance of thyroid hormone. Endocrinology 209:283-297.
19. Jennings, A. S. 1984. Regulation of hepatic triiodothyronine production in the streptozotocin-induced diabetic rat. Am J Physiol. 247:E526-E533.
20. Tabata, S., Nishikawa, M., Toyoda, N., Ogawa, Y., and Inada, M. 1999. Effect of triiodothyronine administration on reduced expression of type 1 iodothyronine deiodinase messenger ribonucleic acid in streptozotocin-induced diabetic rats. Endocr J 46:367-374.

1. A method comprising
a. providing a first control population and a first test population of mammalian cells genetically engineered to express a nucleic acid encoding a deiodinase 1 promoter or ApoA-1 promoter Construct B identified by SEQ ID NO: 24, which promoter is operatively linked to a nucleic acid encoding a reporter protein that can be visualized under conditions that permit the cells in the population to express the reporter protein,
b. contacting the first test population with the a test agent,
c. determining the amount of visualized reporter protein in the first control population and the first test population, and
d. if the determined amount in the first test population is higher than the determined amount in the first control population, then identifying the test agent as one that increases the activity of the respective deiodinase 1 promoter or ApoA-1 promoter.

2. The method of claim 1, wherein if the test agent is identified as one that increases the activity of either the deiodinase 1 promoter or ApoA-1 promoter B construct, then
e. providing a second control and a second test population of the cells that have been transfected with a nucleic acid encoding deiodinase 1 or ApoA-1 protein or a biologically active fragment or variant that has at least 70% sequence identity therewith, which encoding nucleic acid is operatively linked to reporter a reporter protein that can be visualized under conditions that permit the cells to express the reporter protein,
f. contacting the second test population with the test agent,
g. determining the amount of visualized reporter protein in the second control population and the second test population, and
h. if the determined amount in the second test population is higher than the determined amount in the second control population, then identifying the test agent as one that increases deiodinase 1 or ApoA-1 protein expression by increasing activity of the respective promoter.

3. The method of claim 1, wherein the provided control and test populations exhibit reduced insulin receptor expression or biological activity.

4. The method of claim 2, wherein the provided control and test populations exhibit reduced insulin receptor expression or biological activity.

5. The method of claim 1, wherein the reporter protein is a fluorescent protein.

6. The method of claim 5, wherein the fluorescent protein is member selected from the group comprising luciferase, green fluorescent protein, yellow fluorescent protein, blue fluorescent protein, Cerulean fluorescent protein, Cyan fluorescent protein, red fluorescent protein from Zooanthus, red fluorescent protein from Entremacaea quadricolor, luxAB Bioreporters, luxCDABE Bioreporters, Aequorin, and Uroporphyrinogen (urogen) III methyltransferase (UMT).

7. The method of claim 1, wherein the reporter protein is a member selected from the group comprising alkaline phosphatase, horseradish peroxidase, urease, beta galactosidase, and chloramphenicol acyltransferase.

8. The method of claim 3, wherein the insulin receptor gene in the cells has been knocked out.

9. The method of claim 3, wherein the cells in the first control and first test populations are contacted with an oligonucleotide inhibitor of insulin receptor gene transcription or mRNA translation, which inhibitor is sufficiently complementary to the insulin receptor gene or to mRNA encoding the insulin receptor that it reduces transcription or translation, respectively.

10. The method of claim 2,
wherein the test agent is identified as one that increases either deiodinase 1 expression or ApoA-1 expression, then
(i) providing a test animal,
(j) determining a control level of high density lipoprotein cholesterol (HDLC) or ApoA-1 in a first biological sample taken from the animal,
(k) administering the test agent to the test animal,
(l) determining the level of HDLC or ApoA-1 in a second sample taken from the animal at a prescribed time after administering the test agent, and
(m) if the level of HDLC level or ApoA-1 in the second sample is higher than the respective level in the first sample, then identifying the test agent as one that increases HDLC or ApoA-1 levels in the animal.

11. The method of claim 1, wherein the ApoA-1 promoter operatively linked to a reporter protein is a reporter construct pGL3-ApoA-1-LUC.

12. The method of claim 1, wherein the mammalian cells are liver cells.

13. The method of claim 12, wherein the liver cells are from human hepatoma cell line HepG2 or rat hepatoma cell line McARH7777.

14. The method of claim 1, wherein the biological sample is a blood sample, plasma or a tissue sample.

15. A method comprising
identifying a subject with low levels of plasma high density lipoprotein cholesterol (HDLC) and/or plasma ApoA-1 levels, and
administering to the subject deiodinase 1, or a biologically active protein or variant that has at least 70% identity with the amino acid sequence of deiodinase 1, in a therapeutically effective amount that increases the plasma levels of HDLC.

16. The method of claim 15, wherein the deiodinase 1 is human deiodinase for an isoform thereof, identified by an amino acid sequence selected from the group comprising NP_{—}000783, NP_{—}001034804, NP_{—}001034805 and NP_{—}998758.

17. The method of claim 15, wherein the subject is an animal having type 2 diabetes, cardiovascular disease or a disorder associated with impaired or defective insulin signaling.

18. The method of claim 15, wherein the deiodinase 1 is formulated to optimize delivery to the liver.

19. A pharmaceutical formulation comprising human deiodinase 1 or a biologically active protein or variant that has at least 70% identity with the amino acid sequence of deiodinase 1, formulated in liposomes and targeted to the liver.

20. An oligonucleotide identified by SEQ ID NO: 24.


SEQUENCE LISTING (26 sequences)

SEQ ID NO: 1 - DNA, 19 bp, Artificial Sequence
  Synthetic primer murine (m)-Deiodinase 1
cccctggtgttgaactttg

SEQ ID NO: 2 - DNA, 18 bp, Artificial Sequence
  Synthetic primer murine (m)-Deiodinase 1
tgtggcgtgagcttcttc

SEQ ID NO: 3 - DNA, 20 bp, Artificial Sequence
  Synthetic primer mApo-AI
tgtgtatgtggatgcggtca

SEQ ID NO: 4 - DNA, 20 bp, Artificial Sequence
  Synthetic primer mApo-AI
atcccagaagtcccgagtca

SEQ ID NO: 5 - DNA, 20 bp, Artificial Sequence
  Synthetic primer mApo-AII
aatggtcgcactgctggtca

SEQ ID NO: 6 - DNA, 20 bp, Artificial Sequence
  Synthetic primer mApo-AII
ttggccttctccatcaaatc

SEQ ID NO: 7 - DNA, 20 bp, Artificial Sequence
  Synthetic primer mapoB
atgggaagaaacaggcttga

SEQ ID NO: 8 - DNA, 20 bp, Artificial Sequence
  Synthetic primer mapoB
ttctgtcccacgaattgaca

SEQ ID NO: 9 - DNA, 20 bp, Artificial Sequence
  Synthetic primer mapoE
accgcttctgggattacctg

SEQ ID NO: 10 - DNA, 20 bp, Artificial Sequence
  Synthetic primer mapoE
gctgttcctccagctccttt

SEQ ID NO: 11 - DNA, 19 bp, Artificial Sequence
  Synthetic primer mcyclophilin
ggagatggcacaggaggaa

SEQ ID NO: 12 - DNA, 19 bp, Artificial Sequence
  Synthetic primer mcyclophilin
gcccgtagtgcttcagctt

SEQ ID NO: 13 - DNA, 21 bp, Artificial Sequence
  Synthetic mission siRNA SASI_Rn01_00118857 for Dio1
gauugaaauccguuaauautt

SEQ ID NO: 14 - DNA, 21 bp, Artificial Sequence
  Synthetic mission siRNA SASI_Rn01_00118857 for Dio1
auauuaacggauuucaauctt

SEQ ID NO: 15 - DNA, 20 bp, Artificial Sequence
  Synthetic mission siRNA SASI_Rn01_00118856 for Dio1
cucaugaugaugacgucaat

SEQ ID NO: 16 - DNA, 20 bp, Artificial Sequence
  Synthetic mission siRNA SASI_Rn01_00118856 for Dio1
uugacgucaucaucaugagt

SEQ ID NO: 17 - DNA, 19 bp, Artificial Sequence
  Synthetic primer Rat (r)-Insulin receptor
atgggcttcgggagaggat

SEQ ID NO: 18 - DNA, 20 bp, Artificial Sequence
  Synthetic primer Rat (r)-Insulin receptor
ggatgtccataccagggcac

SEQ ID NO: 19 - DNA, 19 bp, Artificial Sequence
  Synthetic primer rDeiodinase 1
cccctggtgttgaactttg

SEQ ID NO: 20 - DNA, 18 bp, Artificial Sequence
  Synthetic primer rDeiodinase 1
tgtggcgtgagcttcttc

SEQ ID NO: 21 - DNA, 20 bp, Artificial Sequence
  Synthetic primer rapoA-1
cctggatgaattccaggaga

SEQ ID NO: 22 - DNA, 20 bp, Artificial Sequence
  Synthetic primer rapoA-1
tcgctgtagagcccaaactt

SEQ ID NO: 23 - DNA, 65 bp, Artificial Sequence
  Human ApoA-I promoter, Construct A
ccacccgggagacctgcaagcctgcagacactcccctcccgcccccactgaacccttgacccctg

SEQ ID NO: 24 - DNA, 62 bp, Artificial Sequence
  Synthetic primer Construct B
ccctgcagcccccgcagcttgctgtttgcccactctatttgcccagccccagggacagagct

SEQ ID NO: 25 - DNA, 130 bp, Artificial Sequence
  Synthetic primer Construct C
gatccttgaactcttaagttccacattgccaggaccagtgagcagcaacagggccggggctgggcttatcagcctcccagcccagaccctggctgcagacataaataggccctgcaagagctggctgctt

SEQ ID NO: 26 - DNA, 3700 bp, Homo sapiens
  misc_feature (1)..(3700): human DIO1 promoter sequence
ttcgtcgacttgagttcttgaccgttccagttttctcttttttgtcctcccagcttctcttcctgccagaacttccttctccccgacttgcccactcagccagcccagcttgtgaatggctgccagattgctcttctctgagtacataccagctcaaccactttcagcagctcccctctgcatttaggatgaagcccaggctcagccttggattccaggtcctccctggtcaggctctagcttttcttctcaattctacctctgagctccccgccacactcatttctttcggacaaactgttgggccttgtacatctcttgtactttcccttgtctttgcctttgctgacatcggctggtcaagaatgcccttcccctctccatcgtcgtctatatccccctcattcatgtgggtccagctctcctgacaccttgtcctccatgaagccacctcagcttcctacagctaggcatgtgctctctcccttcggctcatggctctctgtctgcacctctccttggacactgctgcttcctgctcagcacctggtacctaagcacaagtcttatttccctgcccagtggagagcctcaggagagggtgtgtgtctgatttatctctggattcctcagcatgctcggcccagggcctagatgcagcaggtagagaaggcacctgaggcagttggtttattccgtgtttttcttgtttttctttttctctttttttttttttttttttttttttgagacagagtctcactctactgcccacgctggagtgtaggggtatgatcacgactcactgcatcctcgatttaccaggctcaagccatcctcccaccttagcctccttagtacctgggacaacaagtgcacaccacaatgctcggctaatttttgcattttttgtagaggtggggtttcaccatgttgcccaggctggtctcgaactcctggactcaagtgatccacccacttcggccttccaaaatgctgggattacaggcatgagccactgtgcctggcctatcctgtgtttttgaaagaatgttctttagaacctaagttccacagatatgctttactatgtagtgttgcctggtcaaagtagttgggaaaccctgaatactatatccccctcctatgcaatttcatgtgcaatttcatgtgcacatgagtgtatgcacatgaggagtttacagttccatagaacagatggaggtaataaacaaatccttacagtcctatgaaatgggggaggctataaaaaaatagaacttttgcctggaggacttggaagttttcctggaggaggtggctctggaactaggtcttgaagaatgagtcagatttttgtagcctgacaaggaaaaagggaagagtgttttagaggggaaggcaggagcttcttttgttttgctgttcattcataattttaaaccacagtgcacaaatgacctcagtttattcaacaaatgttcactaattccattggtagtaagagcaatggtaataactaacttaccacatgcccatgtgccaagcactgtaacagaaccaggccaatttgctgaatgccagtcatctgcagttcagttccctgaaagccagcttgcctcatggccaattcatggaatgtacttgcatcatgtaactgtccactttcagtgaggcagtttacattttaaagactgttgaatttggtctgagccccgtggctcacgcctttaattccagcactttgggaggctgaggcgggcagatcacttgaggtcagaagttcaagaccagcctggccaacatgatgaaacctcgtctctactaaaaatacaaaaatcagccaggcatggtggcatgcacctataatcccagctactcagaaggctgaggcatgagaatcacttgaacccaggaggcagaggttgcagtgagccgagatcgcaccactgcactccagcctgggtgacacagcgagactctgtcttaaaataaaataaaataaaatataaaataaaataaaaactgttcagtttgtctctgctccctgctgctgcagctgagactgaaaattggtaggagtgaccagttgcagtggcccatgcctgtaatcccagcactgtgagcggctgagtgggaggattgcttgaacccaggagttcaagaacagcctgtgcaacagagtggaaccctgtctctacaaaatatttaaaaattagtgggatgaggtggtgtgagcctgtagtcccagctactcaggaggctgagttgggggggtcacttaagcccaggaggtcgaggcttcagtgagccatgttcatgctagtgcactccagcctaggtaacagagttaagaccttgtctcaaaaataaataagtaaataaaattaaaaatttttaatggtaagaggaggggactgaagcaaaagaaaaatctatttgcaaaatagagtttactttcagcacattaacccaaagtcccctgaaatcataggtactaacaatacggaaataaacaccatgggcctctgccctggaaggcctcataactcagagtgagagatggtgtcgtgacagggaagcagagggcactgggggcaggaaccctgttaagagtagggtaaggaggtggccaagggaaagcttcctggaggagaggatggtgtgctgattgtctagggacagtgaaaccttggggtgggtgaggaagaggggaatggaaagcagggcagggcacagaggaggagcagcagaggtctgagatgtggagaagcaacattcagtttggcacaagtggggtcccagaggcaggaaggggtgaaggatgaggctgaaggcatcatcaggaaccagagcttacggggccttgtgtgtcgtagctgcaggttgactttatcctgagagtactggtgagttctggaagggtttccaagagagaagtaaacatgatcagttctgcttattagaaagacattggccgagcatggtggctcacacctgtaatcccagcactttgggaggccgaggccagcgggtcacttgatgtcaggagttcgagaccagcctggccaacctgatgaaatcctgtctctactaaaaatacaaaaattagccgggcatcgtggcatgcgcctgtaatcccagctccttgggaggctgaggcaggagaattgcttgaacccgggaggtggagtttgtagtgagctgagattgcgccactgcactccagcctgggcaacaaagcgagactctgtctcaaaaaaaaaaaaaaaaaaagagacatgttgtaactactttggaaacccaccaggccaccaaaaagctctgttgtatgctttgggtataaactctgaactcagagccagagacagagagacgtgaagaatctttactgataatctaaagcaaccgcttcgtttttgagatgcaaaagtccagagaggtgaatgactcgcttagagtcacacagtgagttcttagaagagccagaactagacttctgactctcagctcgtgcacttgctgctactggatacgacagcaggagctcagggaaactctcagccacctccagccctctgtgcgtccacacacgcacacacacacaatatacacacactcttggacacacacagaacaaaacatcgagtaactggcatggtgtggcagaaggcaagttctggatgatttactttctgga